SEQUENCE LISTING (19 sequences)

SEQ ID NO: 1 - protein, 2383 aa, Homo sapiens
MWWSTLMSILRARSFWKWISTQTVRIIRAVRAHFGGIMDEPSPLAQPLELNQHSRFIIGSVSEDNSEDEISNLVKLDLLEEKEGSLSPASVGSDTLSDLGISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGVVCGALHVADVSLRNSVSNFLHSLERGQVLPAHTLLNTVDVELIYEGVKYVLKVTRQSPNSYVVIMNGSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQLDNPSKVQQAELHTGSLPRIQSTALRGEKLHRVFHYVLDNLVNVMNGYCLPDPFFSSKVKDWVERLMKTLRDPSLPLLELQDIMTSVSGRIPPNVEKSIKKEMAQYASNITSVLCQFPSQQIANILDSHAATLNRKSEREVFFMNTQSIVQLVQRYRSGIRGHMKAVVMDLLRQYLRVETQFQNGHYDKCVFALREENKSDMNTVLNYIFSHAQVTKKNLLVTMLIDQLCGRDPTLTDELLNILTELTQLSKTTNAKVALRARQVLIASHLPSYELRHNQVESIFLSAIDMYGHQFCIENLQKLILSETSIFDVLPNFFYHSNQVVRMAALEVYVRRAYIAYELNSVQHRQLKDNTCVVEFQFMLPTSHPNRGNIPTLNRMSFSSNLNHYGMTHVASVSDVLLDNSFTPPCQRMGGMVSFRTFEDFVRIFDEVMGCFSDSPPQSPTFPEAGHTSLYDEDKVPRDEPIHILNVAIKTDCDIEDDRLAAMFREFTQQNKATLVDHGIRRLTFLVAQKDFRKQVNYEVDRRFHREFPKFFTFRARDKFEEDRIYRHLEPALAFQLELNRMRNFDLTAIPCANHKMHLYLGAAKVEVGTEVTDYRFFVRAIIRHSDLVTKEASFEYLQNEGERLLLEAMDELEVAFNNTNVRTDCNHIFLNFVPTVIMDPSKIEESVRSMVMRYGSRLWKLRVLQAELKINIRLTPTGKAIPIRLFLTNESGYYLDISLYKEVTDSRTAQIMFQAYGDKQGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 2 - protein, 2458 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

SEQ ID NO: 3 - protein, 774 aa, Artificial Sequence
  Description of Artificial Sequence Synthetic polypeptide
MHHHHHHVEDYKDDDDKENLYFQGSLQAKRFQAQTLGTTYIYDFPEMFRQALFKLWGSPDYYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLS

SEQ ID NO: 4 - protein, 806 aa, Artificial Sequence
  Description of Artificial Sequence Synthetic polypeptide
MHHHHHHVEDYKDDDDKENLYFQGSGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 5 - protein, 774 aa, Artificial Sequence
  Description of Artificial Sequence Synthetic polypeptide
MHHHHHHVEDYKDDDDKENLYFQGSLQAKRFQAQTLGTTYIYDFPEMFRQALFKLWGSPAAYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLS

SEQ ID NO: 6 - protein, 751 aa, Artificial Sequence
  Description of Artificial Sequence Synthetic polypeptide
GSLQAKRFQAQTLGTTYIYDFPEMFRQALFKLWGSPAAYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLS

SEQ ID NO: 7 - protein, 6 aa, Artificial Sequence
  Description of Artificial Sequence Synthetic 6x His tag
HHHHHH

SEQ ID NO: 8 - protein, 560 aa, Artificial Sequence
  Description of Artificial Sequence Synthetic polypeptide
GAAQAKRFQAQTLGTTYIYDFPEMFRAALAALWGAPAAAPADILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIAHMFHVAWAAAAAAAAGPKYLYAAPADAAAAAAAAAAHCAHAAEGGAAAAMATDIAGKDDGAGVENLRGSGMAAGESSLAYEEIVTISLVTCRAIGIGAYLVRLGQRVIQVENSHIILTGASALNAVLGREVYTSNNQLGGVQIMHYNGVSHITVPDDFEGVYTILEWLSYMPKDNHSPVPIITPTDPIDREIEFLPSAAPYDPRWMLAGRPHPTLAGTWQSGFFDHGSFKEIMAPWAQTVVTGRARLGGIPVGVIAVETRTVEVAVPADPAALDAAAAIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADAESRGGVLEPEGTVEIKFRAKDLIASMRRADPAAKAAA

SEQ ID NO: 9 - protein, 126 aa, Artificial Sequence
  Description of Artificial Sequence Synthetic polypeptide
AGAAAAAADAAAPAYHQVAVQFADFHDTPGRMLEKGVISDILAWAAARTFLYWRLRRLLLEDQVKAEILAASGAAAHAAAQSMAAAAFAAAEGAAAAYAAAANAAAAQAAAAAAAAAAAAAAAAAA

SEQ ID NO: 10 - protein, 558 aa, Artificial Sequence
  Description of Artificial Sequence Synthetic polypeptide
GAAAAARFQAATLGTTYIYDFPEMFRQALFKLWGAPAAAAADIATYTELVLDSQGQLVEMNRLPGGNEVGMVAFKMRFKTQEYPEGRDVIVIGNDITFRIGSFGPGEDLLYLRASEMARAEGIPKIYVAANAGARIGMAEEAKHMFHVAWVAPAAPAKGFAYLYLTPQDAAAAAAAAAAAAAHAAAGGASRYMITDIIGKDDGLGVENLRGSGMIAGESSLAYEEIVTISLVTCRAIGIGAYLVRLGQRVIQVEASHIILTGASALNAVLGRAVYTSNNQLGGVQIMHYNGVSHITVPDDFEGVYTILEWLSYMPKDNHSPVPIITPADPIDREIEFAPSRAPYDPRWMLAGRPHPTAAGTWQSGFFDHGSFAEIMAPWAQTVVTGRARAGGIPVGVIAVETRTVEVAVPADPANLDSAAAAIQQAGQVWFPDSAYKTAQAIKDFNREKLPLMIFANWRGFSGGMKDMYDQVLKFGAYIVDGLRQYKQPIAIYIPPYAELRGGSWVVIDATINPLCIEMYADAESRGGVLEPAGTVEIKFRAADLAKSMRRIAPAYKA

SEQ ID NO: 11 - protein, 123 aa, Artificial Sequence
  Description of Artificial Sequence Synthetic polypeptide
AAAAAAALAAPAYHQVAVAFADFHDTPGRMLEAGVISDILAWKTARTFLYWRLRRLLLEDQVKQEILQASGELSHVHIQSMLARWFAEAAGAAAAAAAAAAAAAAAAAAAAAAAAAAAAAAAA

SEQ ID NO: 12 - protein, 564 aa, Artificial Sequence
  Description of Artificial Sequence Synthetic polypeptide
GAAAAARAAAAALGAAYIYDFPEMFRAALAAAWGAPAAAPAAAATYTEAVADSAGQLAAMAAAPGGNAAGMVAFKMRFAAAEYPEGRDAIAAGNDITFRAGSFGPGEDLAYARASEMARAEGAPKAYVAANSGARAGMAEEAAHMAHAAWAAAAAAAAGAKYAYAAPADAAAAAAAAAAAAAAAAAGGAAAAMIAAIAGKADGAGVENLAGSGMIAGESSLAYEEIVTISLVACRAAGIGAYLVRLGQRVIQVEASHIIAAGASALNAVAGAEVYTSNNQLGGAQIMHYNGASHATAPDAFAGVYTILEWLSYMPKDNHAPAPIATPTDPAARAAAFAPSAAPYDPRWMLAGRPHPTAAGTWQSGFFDHGSFKEIMAPWAQTVATGRARLGGIPAGVIAAETRTVEVAVPADPAALAAAAAAIAQAGQAWFPDSAYKTAQAIKDFNREALPLMIFANWRGFSGGMKDMYDQVLKFGAYIVDGLRQYAQPILIYIPPYAALRGGSWVVIDATINPLCAEMYADAASRGGVLEPAGTVEIKFRAADLIASMRRIDPAAAAAAAAAG

SEQ ID NO: 13 - protein, 122 aa, Artificial Sequence
  Description of Artificial Sequence Synthetic polypeptide
AAAAAAAALALAAYHAVAVQFADFHDTPGRMLEKGVISDILEWAAARTFLYWRLRRLLLEDQVKQEALAASGAAAHAAAQAMAAAWFAAAEGAAAAAAAAANAAAAQAAAAAAAAAAAAAAA

SEQ ID NO: 14 - protein, 557 aa, Artificial Sequence
  Description of Artificial Sequence Synthetic polypeptide
GAAAAARFQAQTLGTTYIYDFPEMFRQALFKLWGAPAAAAADIATYTELVLDSQGQLVEMNRAPGGNEVGMVAFKMRFKTQEYPEGRDVAVIGNDITFRAGSFGPGEDLLYLRASEMARAEGIPKIYVAANSGARIGMAAEIKHMFHVAWAAPEAPAAGFAYLYLTPQDYAAASSAAAVAAAAAAEGGAARYMITDIIGKDDGLGVENLRGSGMAAGESSLAYEEIVTISLVTCRAIGIGAALVALGQRVIQVEASHIILTGASALNAVLGRAAYTSNNQLGGVQIMHYNGVSHITVPDDFEGVYTILEWLSYMPKDNHSPAPIITAADPIDREAEAAPSAAPYDPRWMLAGRPHPTAKGAWQSGFFDHGSFAEIMAPWAQTVVTGRARLGGIPVGVIAVETRTVEAAVPADPANLDSAAAAIAQAGQVWFPDSAYKTAQAIKDFNREALPLMIFANWRGFSGGMKDMYDQVLKFGAYIVDGLRQYKQPILIYIPPYAELRGGSWVVIDATINPLCIEMYADAESRGGVLEPAGTAEAAFAAADAAASMRRIAAAAA

SEQ ID NO: 15 - protein, 102 aa, Artificial Sequence
  Description of Artificial Sequence Synthetic polypeptide
ALAAPAAAQAAAQFADAHDTPGRMLAAGAASDIAAWKAARTFLYWRARRLLAEDQVAQEILQASGAASAVHAQAMLAAAAAAAAGAAAAAAAAAAAAAAAAA

SEQ ID NO: 16 - protein, 749 aa, Homo sapiens
  MOD_RES (321)..(330): Any amino acid
LQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVXXXXXXXXXXREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLS

SEQ ID NO: 17 - protein, 757 aa, Homo sapiens
LQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILS

SEQ ID NO: 18 - protein, 740 aa, Saccharomyces cerevisiae
LQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTL

SEQ ID NO: 19 - protein, 799 aa, Artificial Sequence
  Description of Artificial Sequence Synthetic polypeptide
MHHHHHHVEDYKDDDDKENLYFQGSGSQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

(19) United States
(12) Patent Application Publication
(10) Pub. No.: US 2009/0155815 A1
(43) Pub. Date: Jun. 18, 2009

(54) CRYSTAL STRUCTURE OF THE CARBOXYL TRANSFERASE DOMAIN OF HUMAN ACETYL-COA CARBOXYLASE 2 PROTEIN (ACC2 CT) AND USES THEREOF

(76) Inventors: Bruce L. Grasberger, Trappe, PA (US); Cynthia Milligan, Rutledge, PA (US); John C. Spurlino, Downington, PA (US); Ruth A. Steele, West Chester, PA (US); Kenneth R. Singleton, Downington, PA (US); Alan C. Gibbs, Wyndmoor, PA (US); Francis A. Lewandowski, Washington Crossing, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/258,037

(22) Filed: Oct. 24, 2008

Related U.S. Application Data
(60) Provisional application No. 60/982,751, filed on Oct. 26, 2007.

(51) Int. Cl. G01N 33/566; C12N 11/02

(52) U.S. Cl. 435/7.8; 435/177

(57) ABSTRACT

A crystallized human ACC2 CT protein as well as a description of the X-ray diffraction pattern of the crystal are disclosed. The diffraction pattern allows the three dimensional structure of human ACC2 CT to be determined at atomic resolution so that ligand binding sites on human ACC2 CT can be identified and the interactions of ligands with human ACC2 CT amino acid residues can be modeled. Models prepared using such maps permit the design of ligands which can function as active agents which include, but are not limited to, those that function as inhibitors of human ACC2 and human ACC1 proteins.

### CROSS REFERENCE TO RELATED APPLICATIONS

This application claims priority to Application No. 60/982,751 filed on Oct. 26, 2007, the entire contents of which are incorporated by reference herein.

### TECHNICAL FIELD

The present invention generally pertains to the fields of molecular biology, protein crystallization, X-ray diffraction analysis, three-dimensional structural determination, molecular modelling, and structure based rational drug design. The present invention provides a crystallized dimer of the carboxyl transferase domain of human acetyl-CoA carboxylase 2 protein (ACC2 CT) as well as descriptions of the X-ray diffraction patterns. The X-ray diffraction patterns of the crystal in question are of sufficient resolution so that the three-dimensional structure of ACC2 CT can be determined at atomic resolution, ligand binding sites on ACC2 CT can be identified, and the interactions of ligands with amino acid residues of ACC2 CT can be modelled.

The high resolution maps provided by the present invention and the models prepared using such maps also permit the design of ligands which can function as active agents. Thus, the present invention has applications to the design of active agents which include, but are not limited to, those that find use as inhibitors of human acetyl-CoA carboxylase 2 and human acetyl-CoA carboxylase 1.

### BACKGROUND OF THE INVENTION

Various publications, which may include patents, published applications, technical articles and scholarly articles, are cited throughout the specification in parentheses, and full citations of each may be found at the end of the specification. Each of these cited publications is incorporated by reference herein, in its entirety.

Human acetyl-Co carboxylase 1 (ACC1) and human acetyl-Co carboxylase 2 (ACC2) are large multi-functional biotin cofactor enzymes that catalyse the ATP-dependent carboxylation of acetyl-CoA to form malonyl-CoA. The amino acid sequence for full-length human ACC1 is SEQ ID NO: 1 shown in FIG. 1. The amino acid sequence for full-length human ACC2 is SEQ ID NO: 2 shown in FIG. 2. (Abu-Elheiga et al. 1995; Abu-Elheiga et al. 1997) ACC1 is located in the cytoplasm, where the production of malonyl-CoA is the first committed step in fatty acid biosynthesis and the rate limiting reaction for the pathway. ACC2 is located on the surface of the mitochondria, where the malonyl-CoA product controls mitochondrial fatty acid uptake through allosteric inhibition of carnitine palmitoyltransferase I (CPT-I). Thus, ACC1 controls the rate of fatty acid synthesis and ACC2 controls the rate of fatty acid oxidation. Given their crucial roles in fatty acid metabolism, both ACC1 and ACC2 are attractive therapeutic drug targets for the discovery of novel treatments for diabetes, insulin resistance, obesity, and the metabolic syndrome. (Abu-Elheiga et al. 1995; Abu-Elheiga et al. 2000; Abu-Elheiga et al. 2001; Abu-Elheiga et al. 2003; Harwood et al. 2003; Harwood 2004; Harwood 2005; Tong 2005; Tong and Harwood 2006)

The therapeutic potential of targeting ACC2 was dramatically demonstrated with ACC2 knockout mice. The mice were protected from diet-induced diabetes and obesity. Compared to their wild type cohorts, the ACC2 knockout mice had increased muscle fatty acid oxidation, reduced total body fat, reduced body weight, reduced plasma free fatty acids, and reduced plasma glucose. (Abu-Elheiga et al. 2001; Abu-Elheiga et al. 2003) The therapeutic potential of small molecule inhibitors of ACC1 and ACC2 was demonstrated with isozyme-nonselective inhibitors. The inhibitors showed efficacy in rodent models by increasing whole body fatty acid oxidation and reducing both liver and adipose tissue fatty acid synthesis. (U.S. Pat. No. 6,979,741) (Harwood 2004) Design of additional inhibitors would be facilitated by a cocrystal structure of these compounds with the human ACC2 CT protein.

Human ACC2 and human ACC1 have three sub domains, the biotin carboxylase domain (BC), the biotin carboxyl carrier domain (BCC), and the carboxyl transferase domain (CT). The amino acid sequences are 75% identical and 87% homologous for the CT domains of human ACC2 and human ACC1 (FIG. 3). The crystal structure of the yeast homolog of the human ACC2 CT domain has been determined, but the crystal structure of the human protein has not been reported. (U.S. patent application Ser. No. 10/754,687), (Zhang et al. 2003; Zhang et al. 2004) The amino acid sequence of the CT domain of the yeast homolog is only 50% identical and 67% homologous to the human ACC2 CT domain (FIG. 4).

Perhaps owing to the low sequence homology between the yeast and human ACC2 CT domain, a human ACC2 CT domain construct, based on the crystallized yeast construct, did not produce well-behaved protein our labs. In addition, the biological activity for the protein was quite low, when measured with the reverse-coupled NADH enzyme assay. (Guchhait et al. 1974; Polakis et al. 1974; Guchhait et al. 1975) The protein was not suitable for crystallization experiments. The 6H.FLAG.Tev. Human ACC2 1637-2458 construct, referred to as ACC2 Long, produced protein that was mostly aggregated into larger molecular weight species. Only a fraction of the ACC2 Long protein appeared to be a dimer, which is the active form of the yeast enzyme. The yeast ACC CT domain protein was shown to be a dimer in solution, with the active site of the enzyme located at the dimer interface. (U.S. patent application Ser. No. 10/754,687) (Zhang et al. 2003; Zhang et al. 2004; Zhang et al. 2004) The relatively small amount of dimer in the ACC2 Long protein preparation could have explained the low biological activity.

A shorter construct, 6H.FLAG.Tev. Human ACC-2 1685-2422, referred to as ACC2 Short, had regions of both the N-terminus and the C-terminus deleted. The deleted regions were homologous to regions at the N-terminus and the C-terminus of the yeast CT domain protein that were disordered in the crystal structure. Protein produced with the ACC2 Short construct was mostly a monomer. Only a small fraction of the protein appeared to be the appropriate size to be the active dimer and again the biological activity was quite low.

The ACC2 Medium construct, 6H.FLAG.Tev. Human ACC-2 1685-2458, produced protein that was very well behaved. The construct included the N-terminal region of the first ACC2 Long construct, but had the C-terminus deleted like the ACC2 Short construct. ACC2 Medium protein was a homologous dimer by size exclusion chromatography (SEC). In addition, ACC2 Medium protein had significantly more biological activity than protein produced from either the ACC2 Long or ACC2 Short constructs. Chromatograms from SEC and representative examples for enzyme activity of ACC2 Long, ACC2 Short, and ACC2 Medium are shown in FIG. 5.

ACC2 Medium protein was used for high throughput crystallization screening (HTXS). Numerous screens were conducted, including the HTXS_{—}96well_Index crystallization screen at both 22° C. and 4° C. The screens were done with and without compound added to ACC2 Medium protein preparations both with and without the 6HFLAG-tag cleaved. No diffraction quality crystals were produced with ACC2 Medium protein.

Following the disappointing attempts at crystallization, ACC2 Medium protein was analysed using ExSAR's H/D-Ex platform. H/D-Ex is a proprietary hydrogen/deuterium-exchange technology that can be used to characterize the conformational dynamics and structural integrity of a protein. Results from H/D-Ex were used to generate structural data that showed a large flexible region at N-terminus and a small flexible portion at the C-terminus of the ACC2 Medium protein (FIG. 6). The large flexible region at the N-terminus included the 6H.FLAG.Tev portion of the construct as well as a portion of the ACC2 CT domain. A new ACC2 construct was designed using the structural information from ExSAR's H/D-Ex experiments. Compared to the ACC2 Medium construct, the new construct retained the 6H.FLAG.Tev region but had 8 residues deleted from the C-terminus and 17 residues deleted form the N-terminus of the ACC2 CT domain. The new construct was 6H.FLAG.Tev. Human ACC-2 1702-2450 (SEQ ID NO 3: FIG. 7).

In an effort to improve the chances of producing protein that was more amenable to crystallization, alanine or serine substitutions were introduced to alter surface properties of the ACC2 CT protein and promote crystal growth. It has been shown that replacing amino acids having large flexible side chains with smaller residues can lead to X-ray quality crystals of proteins otherwise recalcitrant to crystallization. (Derewenda 2004), The alanine or serine substitutions were targeted to amino acids in turns between regions of H bonded secondary structure based on sequence alignments to the crystallized yeast homolog (U.S. patent application Ser. No. 10/754,687) (Zhang et al. 2003; Zhang et al. 2004; Zhang et al. 2004) and a human homology model (FIG. 8). The substitutions were introduced into the new construct, 6H.FLAG.Tev. Human ACC-2 1702-2450. The un-substituted construct was designated SP2 and the 5 alanine or serine substituted constructs were designated SP2-1 thru SP2-5 (FIG. 9).

As had been done with the ACC2 Long, ACC2 Short, and ACC2 Medium constructs, the new constructs were inserted into a baculovirus expression vector and expressed in insect cells. The SP2-4 construct did not produce any protein, but the reason for the lack of expression was never determined. All of the other new constructs produced protein that retained the improved biophysical properties and improved biological activity of the protein produced with the ACC2 Medium construct (FIG. 10 and FIG. 11). An ACC1 CT domain construct was also designed, expressed, purified, and characterized with SEC and the reverse-coupled enzyme assay. Crystallization screens were not done with the ACC1 construct. The ACC1 CT domain construct is 6H.FLAG.Tev. Human ACC-1 1603-2383. The sequence for the ACC1 CT domain construct is SEQ ID NO 4, shown in FIG. 12. SEC data and the enzyme activity data for the ACC1 construct are shown in FIG. 13.

The purified protein preparations from the 5 new ACC2 constructs were screened with the HTXS_{—}96well_Index crystallization screen. Only one of the constructs produced diffraction quality crystals and the crystals were only obtained for protein prepared with TEV cleavage of the 6H.FLAG-tag. The amino acid sequence for the ACC2 1637-2458 (D1736A, K1737A) construct is SEQ ID NO 5, shown in FIG. 14. The amino acid sequence for the protein after TEV cleavage is SEQ ID NO 6, shown in FIG. 15.

### SUMMARY OF THE INVENTION

The present invention includes methods of producing and using three-dimensional structure information derived from the crystal structure of a dimer of the carboxyl transferase domain of human acetyl-CoA carboxylase 2 protein (ACC2 CT). The present invention also includes specific crystallization conditions to obtain crystals of the inhibitor-ACC2 CT complex. The crystals are subsequently used to obtain a 3-dimensional structure of the complex using X-ray crystallography. The obtained data is used for rational drug discovery with the aim to design compounds that are better inhibitors of human acetyl-CoA carboxylase 2 or human acetyl-CoA carboxylase 1.

The present invention includes a crystal comprising a dimer of the carboxyl transferase domain of human acetyl-CoA carboxylase 2 (ACC2 CT), or a fragment, or target structural motif or derivative thereof, and a ligand, wherein the ligand is a small molecule inhibitor. In another embodiment, the crystal has a spacegroup of P2₁2₁2₁.

In another aspect of the invention, the present invention includes a crystal comprising human ACC2 CT which comprises a peptide having at least 95% sequence identity to SEQ ID NO: 6.

In another aspect of the invention, the invention includes a computer system comprising: (a) a database containing information on the three dimensional structure of a crystal comprising human ACC2 CT, or a fragment or a target structural motif or derivative thereof, and a ligand, wherein the ligand is a small molecule inhibitor, stored on a computer readable storage medium; and, (b) a user interface to view the information.

The present invention also includes a method of evaluating the potential of an agent to associate with ACC CT comprising: (a) exposing ACC CT to the agent; and (b) detecting the association of said agent to ACC CT amino acid residues A459-A462, A530-A538, B261-B270 thereby evaluating the potential of the agent.

The invention further includes a method of evaluating the potential of an agent to associate with the peptide having SEQ ID NO: 6, comprising: (a) exposing SEQ ID NO: 6 to the agent; and (b) detecting the level of association of the agent to SEQ ID NO: 6, thereby evaluating the potential of the agent.

Further included in the present invention is a method of identifying a potential agonist or antagonist against human acetyl-CoA carboxylase comprising: (a) employing the three dimensional structure of ACC2 CT cocrystallized with a small molecule inhibitor to design or select said potential agonist or antagonist.

The invention comprises a method of locating the attachment site of an inhibitor to human acetyl-CoA carboxylase, comprising: (a) obtaining X-ray diffraction data for a crystal of ACC2 CT; (b) obtaining X-ray diffraction data for a complex of ACC2 CT and an inhibitor; (c) subtracting the X-ray diffraction data obtained in step (a) from the X-ray diffraction data obtained in step (b) to obtain the difference in the X-ray diffraction data; (d) obtaining phases that correspond to X-ray diffraction data obtained in step (a); (e) utilizing the phases obtained in step (d) and the difference in the X-ray diffraction data obtained in step (c) to compute a difference Fourier image of the inhibitor; and, (f) locating the attachment site of the inhibitor to ACC2 CT based on the computations obtained in step (e).

The present invention further comprises a method of obtaining a modified inhibitor comprising: (a) obtaining a crystal comprising ACC2 CT and an inhibitor; (b) obtaining the atomic coordinates of the crystal; (c) using the atomic coordinates and one or more molecular modelling techniques to determine how to modify the interaction of the inhibitor with ACC2 CT; and, (d) modifying the inhibitor based on the determinations obtained in step (c) to produce a modified inhibitor.

In another aspect of the invention, the invention includes an isolated protein fragment comprising a binding pocket or active site defined by structure coordinates of ACC CT amino acid residues A459-A462, A530-A538, B261-B270.

In another aspect of the invention, the invention includes an isolated nucleic acid molecule encoding the fragment which comprises a binding pocket or active site defined by structure coordinates of ACC CT amino acid residues A459-A462, A530-A538, B261-B270. In another aspect of the invention, the invention includes a method of screening for an agent that associates with ACC CT, comprising: (a) exposing a protein molecule fragment to the agent; and (b) detecting the level of association of the agent to the fragment. In another aspect of the invention, the invention includes a kit comprising a protein molecule fragment.

The invention additionally comprises a method for the production of a crystal complex comprising a ACC2 CT polypeptide-ligand comprising: (a) contacting the ACC2 CT polypeptide with said ligand in a suitable solution comprising 10% PEG 3350, 100 mM Hepes pH 7.5, 200 mM Proline; and, b) crystallizing said resulting complex of ACC2 CT polypeptide-ligand from said solution.

The invention further includes a method for the production of a crystal comprising ACC2 CT and a ligand wherein the ligand is a small molecule inhibitor comprising crystallizing a peptide comprising the sequence of SEQ ID NO: 6 with a potential inhibitor.

The invention includes a method for identifying a potential inhibitor of human acetyl-CoA carboxylase comprising: a) using a three dimensional structure of ACC2 CT as defined by atomic coordinates according to Table 1; b) replacing one or more ACC2 CT amino acids selected from A459-A462, A530-A538, B261-B270 in said three-dimensional structure with a different amino acid to produce a modified ACC2 CT; c) using said three-dimensional structure to design or select said potential inhibitor; d) synthesizing said potential inhibitor; and, e) contacting said potential inhibitor with said modified ACC2 CT in the presence of a substrate to test the ability of said potential inhibitor to inhibit ACC2 CT or said modified ACC2 CT. Also included in the invention is an inhibitor identified by the method.

### BRIEF DESCRIPTION OF THE DRAWINGS

A preferred embodiment of the present invention will now be described, by way of an example only, with reference to the accompanying drawings wherein:

FIG. 1: SEQ ID NO: 1: Amino acid sequence of Full-length ACC1: Shown is the full-length sequence of human ACC1 (gi:38679960, NP_{—}942131.1).The full-length protein is 2383 amino acids.

FIG. 2: SEQ ID NO: 2: Amino acid sequence of Full-length ACC2: Shown is the full-length sequence of human ACC2 (gi:61743950, NP_{—}001084.2). The full-length ACC2 protein is 2450 amino acids.

FIG. 3: Amino acid sequence alignment for Human ACC2 CT vs. Human ACC1 CT: Shown is the amino acid sequence alignment for the CT domains of the human ACC2 and human ACC1 proteins. The sequences were aligned with BLASTP 2.2.14, from The National Center for Biotechnology Information. The amino acid sequences were taken from the full-length sequences of Human ACC2 (gi:61743950, NP_{—}001084.2) and Human ACC1 (gi:38679960, NP_{—}942131.1). The aligned sequences include 749 amino acids (1702-2450) of ACC2 and 764 amino acids (1620-2450) of ACC1. Query refers to the ACC2 sequence and Sbjct refers to the ACC1 sequence. Human ACC1 CT domain is 75% identical and 87% homologous to the human ACC1 CT domain.

FIG. 4: Amino acid sequence alignment for Human ACC2 CT vs. Yeast ACC CT: Shown is the amino acid sequence alignment for the CT domains of the human ACC2 and yeast ACC proteins. The sequences were aligned with BLASTP 2.2.14, from The National Center for Biotechnology Information. The amino acid sequences were taken from the full-length sequences of human ACC2 (gi:61743950, NP_{—}001084.2) and yeast (Saccharomyces cerevisiae) ACC CT (gi:6324343, NP_{—}014413.1) The aligned sequences include 749 amino acids (1702-2450) of ACC2 and 740 amino acids (1493-2232) of yeast ACC. Query refers to the human ACC2 sequence and Sbjct refers to the yeast ACC sequence. Human ACC2 CT domain is 50% identical and 67% homologous to the yeast ACC CT domain.

FIG. 5: Size Exclusion Chromatography (SEC) results and representative enzyme activity for ACC2 Long, ACC2 Medium, and ACC2 Short: Shown are the results for SEC and the reverse-coupled enzyme assay for the 3 ACC2 CT constructs that are referred to as ACC2 Long, ACC2 Medium, and ACC2 Short. The enzyme assay was done under identical conditions with 0.17 mg/ml for all three samples. ACC2 Long was too long and produced mostly large molecular weight aggregated protein; ACC2 Short was too short and produced protein that was mostly a monomer; and ACC2 Medium produced protein that was a homogeneous dimer with more activity than either the ACC2 Long or ACC2 Short proteins.

FIG. 6: H/D-Ex patterns of ACC2 Medium protein: Shown is an H/D-Ex Profile of ACC2 Medium at 4° C. at pH 7.0. Each block represents peptide analyzed. Each block contains four time points, 15, 50, 150, and 500 seconds from top to bottom. The deuteration level at each time point at each segment is color-coded based on the % deuteration level. The key for % deuteration level is shown below the figure. The high-resolution structural data shows a large flexible region at the N-terminus and a small flexible portion at the C-terminus of the ACC2 Medium protein.

FIG. 7: SEQ ID NO 3: Sequence of 6H.FLAG.Tev. Human ACC-2 1702-2450: Shown is the sequence for the un-substituted construct that was designed based on ExSAR's H/D EX results. The numbering in the figure refers to the amino acid sequence for the human full-length ACC2 protein. The 6H.FLAG.Tev sequence is shown as bold text in capital letters. Aspartic acid 1736 (D) and tyrosine 1737 (Y) are also shown as bold text in capital letters.

FIG. 8: Human ACC2 CT homology model colorized based on ExSAR H/D EX with side chains of amino acids to be substituted shown in white: Shown is a single monomer from the human ACC2 CT homology model colorized based on ExSAR's H/D EX results with amino acid side chains shown in white for residues that were targeted for alanine or serine substitutions.

FIG. 9: List of constructs based on ExSAR H/D EX results and alanine or serine substitution strategy: Shown are the 6 new constructs designed based on ExSAR's H/D EX results with the ACC2 Medium protein and an alanine or serine substitution strategy to increase the chances of producing a protein that was more amenable to crystallization. The un-substituted construct is referred to as SP2 and the alanine or serine substituted constructs are referred to as SP2-1 thru SP2-5.

FIG. 10: SDS Page and SEC for new constructs based on ExSAR's H/D EX results and an alanine or serine substitution strategy: Shown are SDS Page gels and SEC results of protein preparations of the new truncated ACC2 CT domain constructs. The constructs were designed based on ExSAR's H/D EX results with the ACC2 Medium protein and an alanine or serine substitution strategy that was used to increase the chances of producing a protein that was more amenable to crystallization. The un-substituted construct is designated SP2 and the 5 alanine or serine substituted constructs are designated SP2-1 thru SP2-5. The SP2-4 construct did not produce any protein, but the reason for the lack of expression was never determined. All of the other new constructs produced protein that retained the improved biophysical properties of the ACC2 Medium construct. Based on the SDS PAGE and UV analysis (not shown), all of the protein preparations were approximately 95% pure. Based on SEC, all of the protein preparations were homogeneous dimers.

FIG. 11: Enzyme activity for the new constructs that were designed based on ExSAR's H/D EX results and an alanine or serine substitution strategy: Shown is the reverse-coupled enzyme assay data for protein preparations of the new truncated ACC2 CT domain constructs. The constructs were designed based on ExSAR's H/D EX results with the ACC2 Medium protein and an alanine or serine substitution strategy that was used to increase the chances of producing a protein that was more amenable to crystallization. The un-substituted construct is designated SP2 and the 5 alanine or serine substituted constructs are designated SP2-1 thru SP2-5. The SP2-4 construct did not produce any protein, but the reason for the lack of expression was never determined. All of the other new constructs produced protein that retained the improved biological activity of the ACC2 medium construct. The new ACC2 constructs all had comparable activity. Also shown is the activity of the ACC1 CT domain construct. Note that four times less protein was used for the ACC1 preparation. The activity of the ACC1 preparations were routinely measured to be approximately four times more active than the ACC2 preparations, but the reason for the increased activity was never determined.

FIG. 12: SEQ ID NO: 4: Amino acid sequence of 6H.FLAG.Tev. Human ACC-1 1603-2383: Shown is the amino acid sequence for the 6H.FLAG.Tev. Human ACC-1 1603-2383 construct. The numbering in the figure refers to the amino acid sequence for the human full-length ACC1 protein. The 6H.FLAG.Tev sequence is shown as bold text in capital letters.

FIG. 13: SDS PAGE, SEC, and enzyme activity for ACC1 protein produced with the ACC1 CT domain construct, 6H.FLAG.Tev. Human ACC-1 1603-2383: Shown is an SDS PAGE of purified ACC1 CT domain protein produced from the 6H.FLAG.Tev. Human ACC-1 1603-2383 construct. ACC1 protein was approximately 95% pure by SDS PAGE. Also shown are SEC and enzyme assay data comparing ACC1 protein to the ACC2 Medium protein. The SEC chromatograms are shown superimposed for ACC1 and ACC2 Medium. ACC1 was a homogeneous dimer by SEC. The activity of the ACC1 preparations were routinely measured to be approximately four times more active than the ACC2 preparations, but the reason for the increased activity was never determined.

FIG. 14: SEQ ID NO: 5: Amino acid sequence of 6H.FLAG.Tev. Human ACC-2 1702 -2450 (D1736A, K1737A): Shown is the amino acid sequence of the construct used to produce the crystallized protein of the present invention. The construct includes the 6H.FLAG-tag and the Tev cleavage site, which are shown in bold text and as capital letters, the human ACC2 sequence from 1702-2450, and the amino acid substitutions D1736A and K1737A, also shown in bold text and as capital letters. The numbering in the figure refers to the amino acid sequence for the human full-length ACC2 protein.

FIG. 15: SEQ ID NO: 6: Amino Acid Sequence of Crystallized Form of Human ACC2 CT: Shown is the amino acid sequence for the crystallized form of the human ACC2 CT domain protein. The total length of the crystallized form of the protein is 751 amino acids and includes GS, which is left after cleavage of 6H.FLAG-tag at the Tev site, and human ACC2 1702-2450 (D1736A, K1737A). The GS and the alanine substitutions, D1736A and K1737A, are shown in bold text as capital letters. The numbering in the figure refers to the amino acid sequence for the full-length human ACC2 protein.

FIG. 16: Structure: Shown is the structure of the compound used during crystallization of the ACC2 CT domain.

FIG. 17: Ribbon representation of ACC2 CT bound to compound. Shown is a ribbon diagram of the protein structure with monomer A in cyan and monomer B in green, the compound is represented as a magenta stick model.

FIG. 18: Fit of compound into the active site of ACC2 CT represented as a molecular surface. Shown is the accessible surface of the two monomers represented in atom coloring with carbons from monomer A colored in cyan, carbons from monomer B colored magenta, oxygens colored red and nitrogens colored blue. The compound is represented as a stick model with carbons colored green, oxygens red and nitrogens blue.

FIG. 19: Close-up of fit of compound into the active site of ACC2 CT represented as a molecular surface. Shown is the accessible surface of the two monomers represented in atom coloring with carbons from monomer A colored in cyan, carbons from monomer B colored magenta, oxygens colored red and nitrogens colored blue. The compound is represented as a stick model with carbons colored green, oxygens red and nitrogens blue.

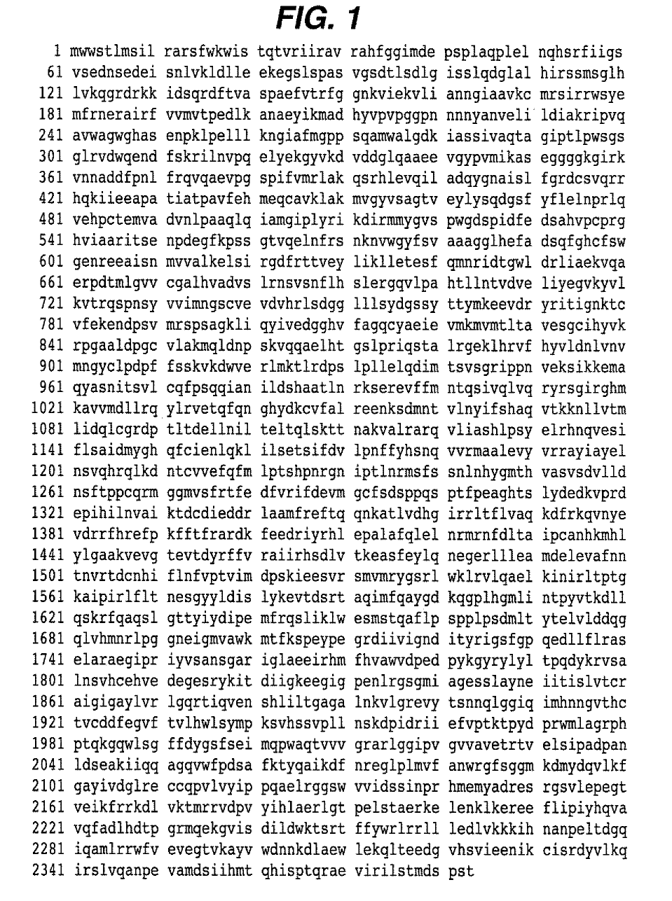

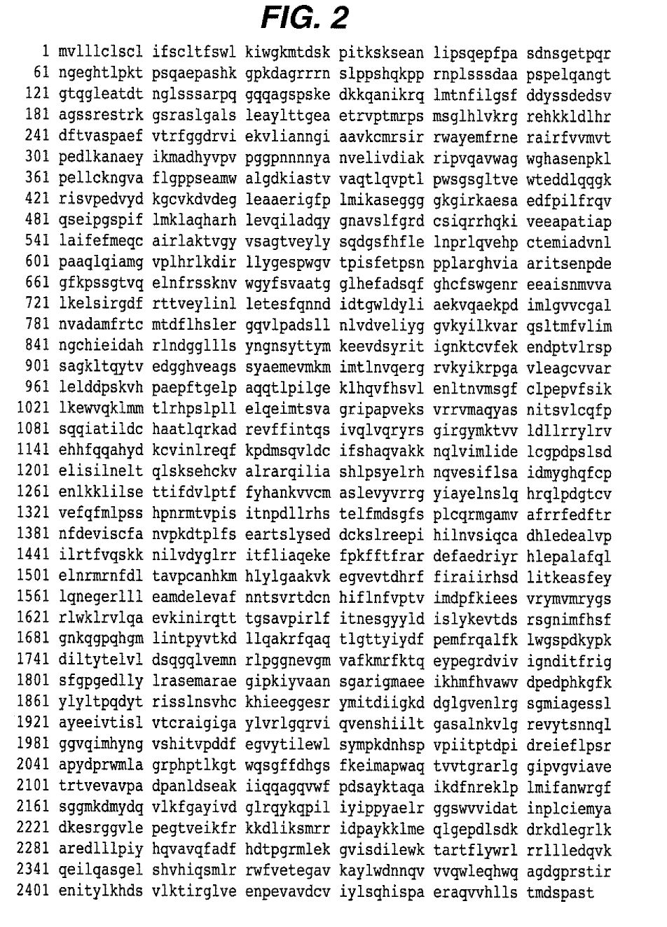

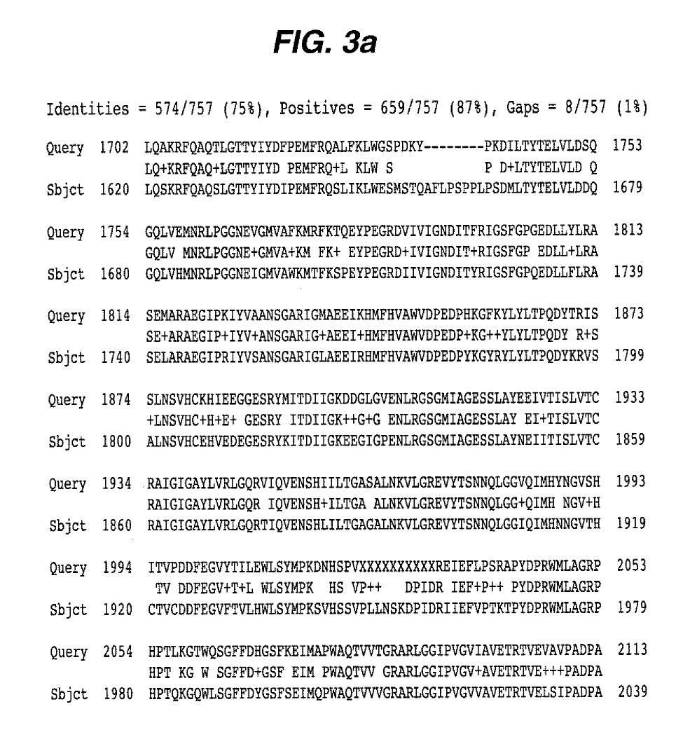

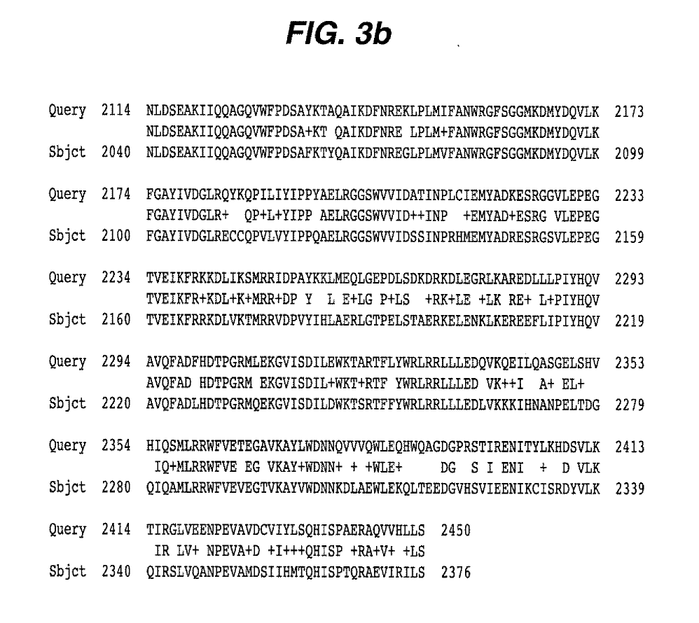

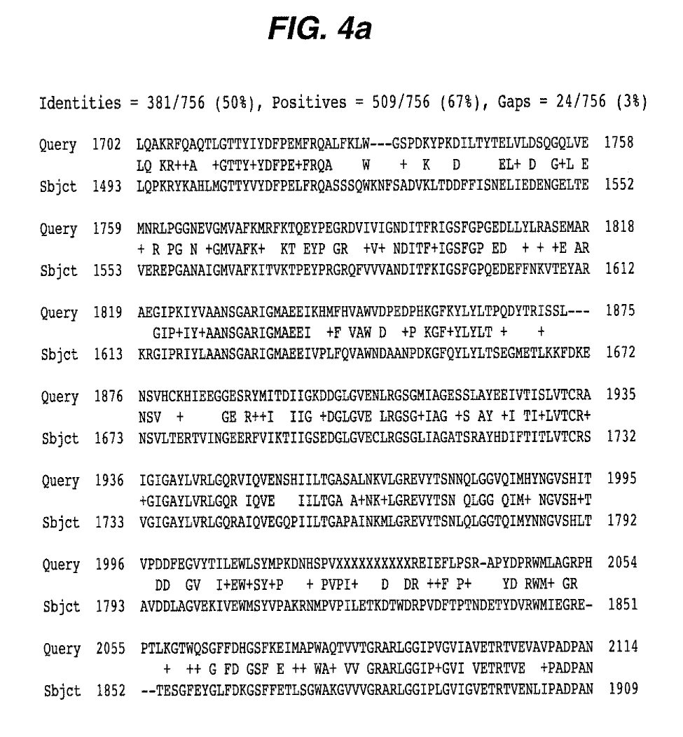

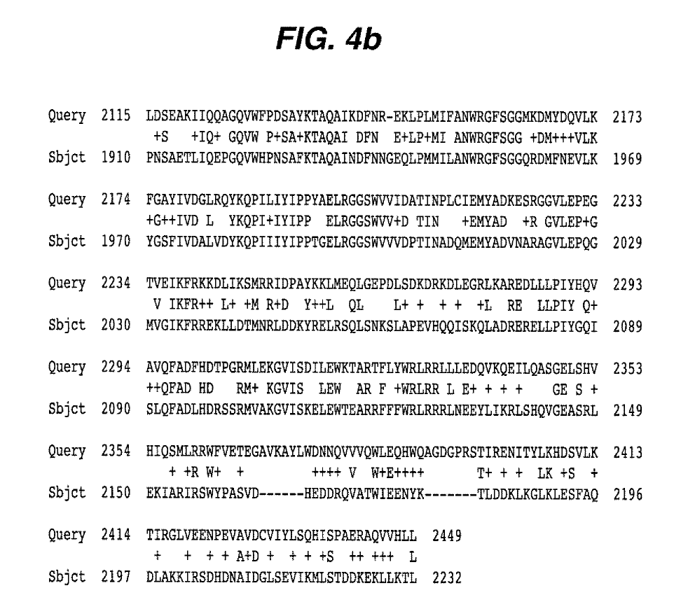

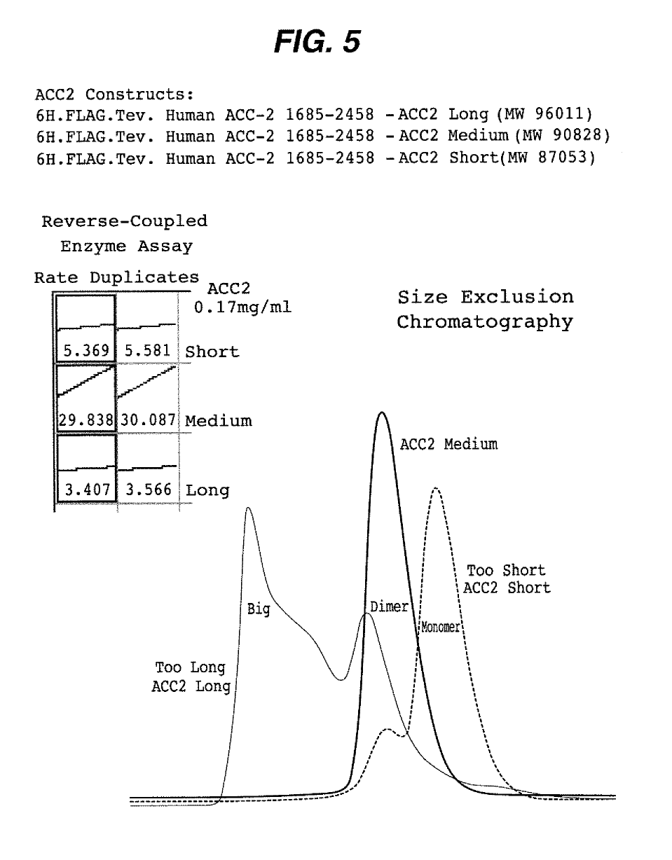

Table: 1: Coordinates for ACC2 CT domain crystal structure in PDB Format. Shown are the coordinates for the structure of ACC2 CT domain in PDB format

### DEFINITIONS

As is generally the case in biotechnology and chemistry, the description of the present invention has required the use of a number of terms of art. Although it is not practical to do so exhaustively, definitions for some of these terms are provided here for ease of reference. Unless defined otherwise, all technical and scientific terms used herein have the same meaning as commonly understood by one of ordinary skill in the art to which this invention belongs. Definitions for other terms also appear elsewhere herein. However, the definitions provided here and elsewhere herein should always be considered in determining the intended scope and meaning of the defined terms. Although any methods and materials similar or equivalent to those described herein can be used in the practice of the present invention, the preferred methods and materials are described.

The term “comprising” means “including principally, but not necessarily solely”. Furthermore, variations of the word “comprising”, such as “comprise” and “comprises”, have correspondingly varied meanings.

As used herein, the term “atomic coordinates” or “structure coordinates” refers to mathematical coordinates that describe the positions of atoms in crystals of ACC2 CT in Protein Data Bank (PDB) format, including X, Y, Z and B, for each atom. The diffraction data obtained from the crystals are used to calculate an electron density map of the repeating unit of the crystal. The electron density maps may be used to establish the positions (i.e. coordinates X, Y and Z) of the individual atoms within the crystal. Those of skill in the art understand that a set of structure coordinates determined by X-ray crystallography is not without standard error. For the purpose of this invention, any set of structure coordinates for ACC2 CT from any source having a root mean square deviation of non-hydrogen atoms of less than about 1.5 Å when superimposed on the non-hydrogen atom positions of the corresponding atomic coordinates of Table 1 are considered substantially identical or homologous. In a more preferred embodiment, any set of structure coordinates for ACC2 CT from any source having a root mean square deviation of non-hydrogen atoms of less than about 0.75 .ANG. when superimposed on the non-hydrogen atom positions of the corresponding atomic coordinates of Table 1 are considered substantially identical or homologous.

The term “atom type” refers to the chemical element whose coordinates are measured. The first letter in a column in Table 1 identifies the element.

The terms “X,” “Y” and “Z” refer to the crystallographically-define-d atomic position of the element measured with respect to the chosen crystallographic origin. The term “B” refers to a thermal factor that measures the mean variation of an atom's position with respect to its average position.

As used herein, the term “crystal” refers to any three-dimensional ordered array of molecules that diffracts X-rays.

As used herein, the term “carrier” in a composition refers to a diluent, adjuvant, excipient, or vehicle with which the product is mixed.

As used herein, the term “composition” refers to the combining of distinct elements or ingredients to form a whole. A composition comprises more than one element or ingredient. For the purposes of this invention, a composition will often, but not always comprise a carrier.

As used herein, “ACC2 CT” is used to mean a protein obtained as a result of expression of the carboxyl transferase domain of the human actyl-CoA carboxylase 2 gene. Within the meaning of this term, it will be understood that human ACC2 CT encompasses all proteins encoded by the carboxyl transferase domain of the human actyl-CoA carboxylase 2, mutants thereof, conservative amino acid substitutions, alternative splice proteins thereof, and phosphorylated proteins thereof. Additionally, as used herein, it will be understood that the term “ACC2 CT” includes the carboxyl transferase domain of human actyl-CoA carboxylase 2, the carboxyl transferase domain of human actyl-CoA carboxylase 1 and homologues of other animals. As an example, ACC2 CT includes the protein comprising SEQ ID NO: 6 and variants thereof comprising at least about 70% amino acid sequence identity to SEQ ID NO: 6, or preferably 80%, 85%, 90% and 95% sequence identity to SEQ ID NO: 6, or more preferably, at least about 95% or more sequence identity to SEQ ID NO: 6.

As used herein, the term “SAR,” an abbreviation for Structure-Activity Relationships, collectively refers to the structure-activity/structure property relationships pertaining to the relationship(s) between a compound's activity/properties and its chemical structure.

As used herein, the term “molecular structure” refers to the three dimensional arrangement of molecules of a particular compound or complex of molecules (e.g., the three dimensional structure of ACC2 CT and ligands that interact with ACC2 CT.

As used herein, the term “molecular modeling” refers to the use of computational methods, preferably computer assisted methods, to draw realistic models of what molecules look like and to make predictions about structure activity relationships of ligands. The methods used in molecular modeling range from molecular graphics to computational chemistry.

As used herein, the term “molecular model” refers to the three dimensional arrangement of the atoms of a molecule connected by covalent bonds or the three dimensional arrangement of the atoms of a complex comprising more than one molecule, e.g., a protein-ligand complex.

As used herein, the term “molecular graphics” refers to 3 D representations of the molecules, for instance, a 3 D representation produced using computer assisted computational methods.

As used herein, the term “computational chemistry” refers to calculations of the physical and chemical properties of the molecules.

As used herein, the term “molecular replacement” refers to a method that involves generating a preliminary model of a crystal of ACC2 CT whose coordinates are unknown, by orienting and positioning the said atomic coordinates described in the present invention so as best to account for the observed diffraction pattern of the unknown crystal. Phases can then be calculated from this model and combined with the observed amplitudes to give an approximate Fourier synthesis of the structure whose coordinates are unknown. (Rossmann 1972)

As used herein, the term “homolog” refers to the ACC2 CT protein molecule or the nucleic acid molecule which encodes the protein, or a functional domain from said protein from a first source having at least about 70% or 75% sequence identity, or at least about 80% sequence identity, or more preferably at least about 85% sequence identity, or even more preferably at least about 90% sequence identity, and most preferably at least about 95%, 97% or 99% amino acid or nucleotide sequence identity, with the protein, encoding nucleic acid molecule or any functional domain thereof, from a second source. The second source may be a version of the molecule from the first source that has been genetically altered by any available means to change the primary amino acid or nucleotide sequence or may be from the same or a different species than that of the first source.

As used herein, the term “active site” refers to regions on ACC2 CT or a structural motif of ACC2 CT that are directly involved in the function or activity of human ACC2 CT.

As used herein, the terms “binding site” or “binding pocket” refer to a region of human ACC2 CT or a molecular complex comprising ACC2 CT that, as a result of the primary amino acid sequence of human ACC2 CT and/or its three-dimensional shape, favourably associates with another chemical entity or compound including ligands, cofactors, or inhibitors.

For the purpose of this invention, any active site, binding site or binding pocket defined by a set of structure coordinates for ACC2 CT or for a homolog of ACC2 CT from any source having a root mean square deviation of non-hydrogen atoms of less than about 1.5 .ANG. when superimposed on the non-hydrogen atom positions of the corresponding atomic coordinates of Table 1 are considered substantially identical or homologous. In a more preferred embodiment, any set of structure coordinates for ACC2 CT or a homolog of ACC2 CT from any source having a root mean square deviation of non-hydrogen atoms of less than about 0.75 .ANG. when superimposed on the non-hydrogen atom positions of the corresponding atomic coordinates of Table 1 are considered substantially identical or homologous.

The tem “root mean square deviation” means the square root of the arithmetic mean of the squares of the deviations from the mean.

As used herein, the term “amino acids” refers to the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxylglutamic acid, arginine, ornithine, and lysine. Unless specifically indicated, all amino acids are referred to in this application are in the L-form.

As used herein, the term “nonnatural amino acids” refers to amino acids that are not naturally found in proteins. For example, selenomethionine.

As used herein, the term “positively charged amino acid” includes any amino acids having a positively charged side chain under normal physiological conditions. Examples of positively charged naturally occurring amino acids are arginine, lysine, and histidine.

As used herein, the term “negatively charged amino acid” includes any amino acids having a negatively charged side chains under normal physiological conditions. Examples of negatively charged naturally occurring amino acids are aspartic acid and glutamic acid.

As used herein, the term “hydrophobic amino acid” includes any amino acids having an uncharged, nonpolar side chain that is relatively insoluble in water. Examples of naturally occurring hydrophobic amino acids are alanine, leucine, isoleucine, valine, proline, phenylalanine, tryptophan, and methionine.

As used herein, the term “hydrophilic amino acid” refers to any amino acids having an uncharged, polar side chain that is relatively soluble in water. Examples of naturally occurring hydrophilic amino acids are serine, threonine, tyrosine, asparagine, glutamine and cysteine.

As used herein, the term “hydrogen bond” refers to two hydrophilic atoms (either O or N), which share a hydrogen that is covalently bonded to only one atom, while interacting with the other.

As used herein, the term “hydrophobic interaction” refers to interactions made by two hydrophobic residues or atoms (such as C).

As used herein, the term “conjugated system” refers to more than two double bonds are adjacent to each other, in which electrons are completely delocalized with the entire system. This also includes and aromatic residues.

As used herein, the term “aromatic residue” refers to amino acids with side chains having a delocalized conjugated system. Examples of aromatic residues are phenylalanine, tryptophan, and tyrosine.

As used herein, the phrase “inhibiting the binding” refers to preventing or reducing the direct or indirect association of one or more molecules, peptides, proteins, enzymes, or receptors, or preventing or reducing the normal activity of one or more molecules, peptides, proteins, enzymes or receptors, e.g., preventing or reducing the direct or indirect association of human ACC2 CT with actyl-CoA or biotin.

As used herein, the term “competitive inhibitor” refers to inhibitors that bind to human ACC2 CT at the same sites as its substrate(s), (e.g., actyl-CoA or biotin), thus directly competing with them. Competitive inhibition may, in some instances, be reversed completely by increasing the substrate concentration.

As used herein, the term “uncompetitive inhibitor” refers to one that inhibits the functional activity of human ACC2 CT by binding to a different site than does its substrate(s) (e.g., actyl-CoA or biotin).

As used herein, the term “non-competitive inhibitor” refers to one that can bind to either the free or actyl-CoA bound form of ACC2 CT.

Those of skill in the art may identify inhibitors as competitive, uncompetitive, or non-competitive by computer fitting enzyme kinetic data using standard methods. See, for example, (Segel 1975)

As used herein, the term “R or S-isomer” refers to two possible stereroisomers of a chiral carbon according to the Cahn-Ingold-Prelog system adopted by International Union of Pure and Applied Chemistry (IUPAC). Each group attached to the chiral carbon is first assigned to a preference or priority a, b, c, or d on the basis of the atomic number of the atom that is directly attached to the chiral carbon. The group with the highest atomic number is given the highest preference a, the group with next highest atomic number is given the next highest preference b; and so on. The group with the lowest preference (d) is then directed away from the viewer. If the trace of a path from a to b to c is counter clockwise, the isomer is designated (S); in the opposite direction, clockwise, the isomer is designated (R).

As used herein, the term “ligand” refers to any molecule, or chemical entity, which binds with or to ACC2 CT, a subunit of ACC2 CT, a domain of ACC2 CT, a target structural motif of ACC2 CT, or a fragment of ACC2 CT. Thus, ligands include, but are not limited to, small molecule inhibitors, for example.

As used herein, the term “small molecule inhibitor” refers to compounds useful in the present invention having measurable ACC2 CT inhibiting activity. In addition to small organic molecules, peptides, antibodies, cyclic peptides and peptidomimetics are contemplated as being useful in the disclosed methods. Preferred inhibitors are small molecules, preferably less than 700 Daltons, and more preferably less than 450 Daltons.

As used herein the terms “bind,” “binding,” “bond,” or “bonded” when used in reference to the association of atoms, molecules, or chemical groups, refer to any physical contact or association of two or more atoms, molecules, or chemical groups.

As used herein, the terms “covalent bond” or “valence bond” refer to a chemical bond between two atoms in a molecule created by the sharing of electrons, usually in pairs, by the bonded atoms.

As used herein, “noncovalent bond” refers to an interaction between atoms and/or molecules that does not involve the formation of a covalent bond between them.

As used herein, the term “native protein” refers to a protein comprising an amino acid sequence identical to that of a protein isolated from its natural source or organism.

### DETAILED DESCRIPTION OF ILLUSTRATIVE EMBODIMENTS

It is to be understood at the outset, that the figures and examples provided herein are to exemplify, and not to limit the invention and its various embodiments.

The present invention includes a crystal comprising the carboxyl transferase domain of human acetyl-CoA carboxylase 2 (ACC2 CT), or a fragment, or target structural motif or derivative thereof, and a ligand, wherein the ligand is a small molecule inhibitor. In one embodiment, the fragment or derivative thereof is a peptide comprising SEQ ID NO: 6

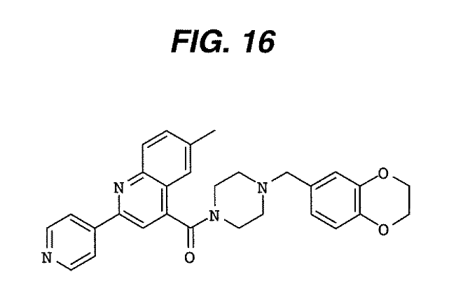

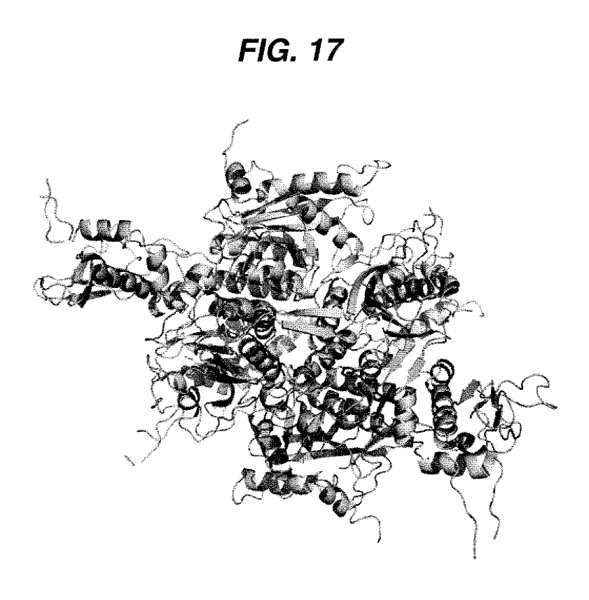

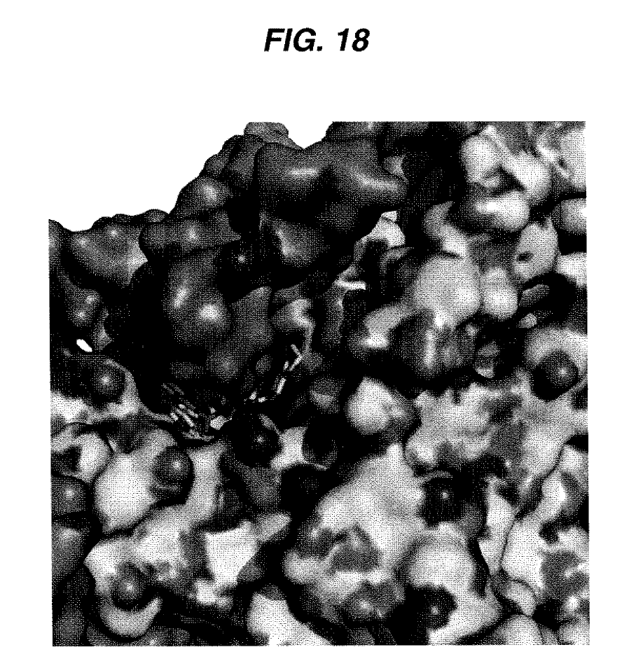

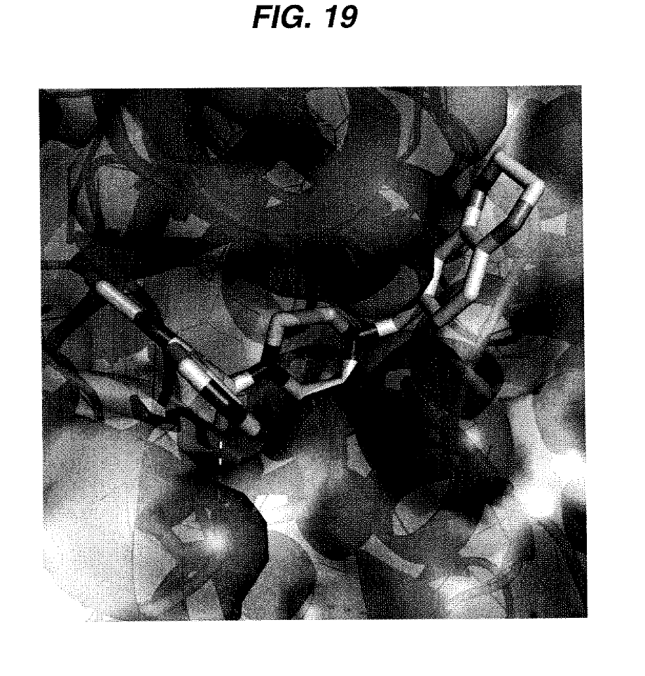

In another embodiment, the crystal has a spacegroup of P2₁2₁2₁. In a different embodiment, the crystal effectively diffracts X-rays for determination of atomic coordinates to a resolution of at least about 3.2 Å. In a preferred embodiment, the ligand is in crystalline form. In a highly preferred embodiment, the ligand is the structure depicted in FIG. 16, and, derivatives thereof.

The present invention also includes a crystal comprising ACC2 CT, which comprises a peptide having at least 95% sequence identity to SEQ ID NO. 2. In a preferred embodiment, the crystal comprising SEQ ID NO: 6 comprises an atomic structure characterized by the coordinates of Table 1. In another preferred embodiment, the crystal comprises a unit cell selected from the group consisting of: a cell having dimensions of a=100.646, b=145.993, c=308.696, alpha=90.00, beta=90.00, gamma=90.00.

In another aspect of the invention, the invention includes a computer system comprising: (a) a database containing information on the three dimensional structure of a crystal comprising ACC2 CT, or a fragment or a target structural motif or derivative thereof, and a ligand, wherein the ligand is a small molecule inhibitor, stored on a computer readable storage medium; and, (b) a user interface to view the information. In one embodiment, the information comprises diffraction data obtained from a crystal comprising SEQ ID NO: 6. In another embodiment, the information comprises an electron density map of a crystal form comprising SEQ ID NO: 6. In a different embodiment, the information comprises the structure coordinates of Table 1 or homologous structure coordinates comprising a root mean square deviation of non-hydrogen atoms of less than about 1.5 A when superimposed on the non-hydrogen atom positions of the corresponding atomic coordinates of Table 1. In a preferred embodiment, the information comprises structure coordinates comprising a root mean square deviation of non-hydrogen atoms of less than about 0.75 Å when superimposed on the non-hydrogen atom positions of the corresponding atomic coordinates of Table 1. In a highly preferred embodiment, the information comprises the structure coordinates for amino acids A459-A462, A530-A538, B261-B270 according to Table 1 or similar structure coordinates for said amino acids comprising a root mean square deviation of non-hydrogen atoms of less than about 1.5 A when superimposed on the non-hydrogen atom positions of the corresponding atomic coordinates of Table 1.

The present invention also includes a method of evaluating the potential of an agent to associate with ACC2 CT comprising: (a) exposing ACC2 CT to the agent; and (b) detecting the association of said agent to ACC2 CT amino acid residues A459-A462, A530-A538, B261-B270 thereby evaluating the potential. In one embodiment of the invention, the agent is a virtual compound. In another embodiment of the invention, step (a) comprises comparing the atomic structure of the compound to the three dimensional structure of ACC2 CT. In a different embodiment, the comparing comprises employing a computational means to perform a fitting operation between the compound and at least one binding site of ACC2 CT. In a preferred embodiment, the binding site is defined by structure coordinates for amino acids A459-A462, A530-A538, B261-B270 according to Table 1 or similar structure coordinates for said amino acids comprising a root mean square deviation of non-hydrogen atoms of less than about 1.5 Å when superimposed on the non-hydrogen atom positions of the corresponding atomic coordinates of Table 1. In a highly preferred embodiment, the agent is exposed to crystalline SEQ ID NO: 6 and the detecting of step (b) comprises determining the three dimensional structure of the agent-SEQ ID NO: 6 complex.

The present invention includes a method of identifying a potential agonist or antagonist against ACC2 CT comprising: (a) employing the three dimensional structure of ACC2 CT cocrystallized with a small molecule inhibitor to design or select said potential agonist or antagonist. In one embodiment, the three dimensional structure corresponds to the atomic structure characterized by the coordinates of Table 1 or similar structure coordinates comprising a root mean square deviation of non-hydrogen atoms of less than about 1.5 Å when superimposed on the non-hydrogen atom positions of the corresponding atomic coordinates of Table 1. In a different embodiment, the method further comprises the steps of: (b) synthesizing the potential agonist or antagonist; and (c) contacting the potential agonist or antagonist with ACC2 CT.

The instant invention comprises a method of locating the attachment site of an inhibitor to ACC2 CT, comprising: (a) obtaining X-ray diffraction data for a crystal of ACC2 CT; (b) obtaining X-ray diffraction data for a complex of ACC2 CT and an inhibitor; (c) subtracting the X-ray diffraction data obtained in step (a) from the X-ray diffraction data obtained in step (b) to obtain the difference in the X-ray diffraction data; (d) obtaining phases that correspond to X-ray diffraction data obtained in step (a); (e) utilizing the phases obtained in step (d) and the difference in the X-ray diffraction data obtained in step (c) to compute a difference Fourier image of the inhibitor; and, (f) locating the attachment site of the inhibitor to ACC2 CT based on the computations obtained in step (e).

The present invention further comprises a method of obtaining a modified inhibitor comprising: (a) obtaining a crystal comprising ACC2 CT and an inhibitor; (b) obtaining the atomic coordinates of the crystal; (c) using the atomic coordinates and one or more molecular modeling techniques to determine how to modify the interaction of the inhibitor with ACC2 CT; and, (d) modifying the inhibitor based on the determinations obtained in step (c) to produce a modified inhibitor. In one embodiment, the crystal comprises a peptide having SEQ ID NO: 6. In a different embodiment, the one or more molecular modeling techniques are selected from the group consisting of graphic molecular modeling and computational chemistry. In a preferred embodiment, step (a) comprises detecting the interaction of the inhibitor to ACC2 CT amino acid residues A459-A462, A530-A538, B261-B270. In another embodiment of the invention, the invention includes an ACC2 CT inhibitor identified by this method.

In another aspect of the invention, the invention includes an isolated protein fragment comprising a binding pocket or active site defined by structure coordinates of ACC2 CT amino acid residues A459-A462, A530-A538, B261-B270. In one embodiment, the isolated fragment is linked to a solid support.

In another aspect of the invention, the invention includes an isolated nucleic acid molecule encoding the fragment, which comprises a binding pocket or active site defined by structure coordinates of ACC2 CT. In one embodiment, a vector comprises the nucleic acid molecule. In another embodiment, a host cell comprises the vector. In yet another aspect of the invention, the invention includes a method of producing a protein fragment, comprising culturing the host cell under conditions in which the fragment is expressed. In another aspect of the invention, the invention includes a method of screening for an agent that associates with ACC2 CT, comprising: (a) exposing a protein molecule fragment to the agent; and (b) detecting the level of association of the agent to the fragment. In another aspect of the invention, the invention includes a kit comprising a protein molecule fragment.

In another aspect of the invention, the invention includes a method for the production of a crystal complex comprising an ACC2 CT polypeptide-ligand comprising: (a) contacting the ACC2 CT polypeptide with said ligand in a suitable solution comprising 10% PEG 3350; 100 mM Hepes pH 7.5; 200 mM Proline; and, b) crystallizing said resulting complex of ACC2 CT polypeptide-ligand from said solution. In one embodiment, the ACC2 CT polypeptide is a polypeptide having SEQ ID NO: 6. In another embodiment, PEG has an average molecular weight range from 2000 to 5000, wherein said PEG is present in solution at a range from about 5% w/v to about 20% w/v and said Proline is present in solution at a range of from about 100 mM to about 300 mM. In a preferred embodiment, PEG has an average molecular weight of about 3350 and is present in solution at about 10% w/v and said Proline is present in solution at about 200 mM.

The invention further includes a method for the production of a crystal comprising ACC2 CT and a ligand wherein the ligand is a small molecule inhibitor comprising crystallizing a peptide comprising SEQ ID NO: 6 with a potential inhibitor.

The invention includes a method for identifying a potential inhibitor of ACC2 CT comprising: a) using a three dimensional structure of ACC2 CT as defined by atomic coordinates according to Table 1; b) replacing one or more ACC2 CT amino acids selected from A459-A462, A530-A538, B261-B270 in said three-dimensional structure with a different amino acid to produce a modified ACC2 CT; c) using said three-dimensional structure to design or select said potential inhibitor; d) synthesizing said potential inhibitor; and, e) contacting said potential inhibitor with said modified ACC2 CT in the presence of a substrate to test the ability of said potential inhibitor to inhibit ACC2 CT or said modified ACC2 CT. In another embodiment, the potential inhibitor is selected from a database. In a preferred embodiment, the potential inhibitor is designed de novo. In another preferred embodiment, the potential inhibitor is designed from a known inhibitor. In a highly preferred embodiment, the step of employing said three-dimensional structure to design or select said potential inhibitor comprises the steps of: a) identifying chemical entities or fragments capable of associating with modified ACC2 CT; and b) assembling the identified chemical entities or fragments into a single molecule to provide the structure of said potential inhibitor. In one embodiment, the potential inhibitor is a competitive inhibitor of SEQ ID NO: 6. In a different embodiment, the potential inhibitor is a non-competitive or uncompetitive inhibitor of SEQ ID NO: 6. In yet another embodiment, an inhibitor is identified by the method.

### A. Modeling the Three-Dimensional Structure of ACC2 CT

The atomic coordinate data provided in Table 1, or the coordinate data derived from homologous proteins may be used to build a three-dimensional model of ACC2 CT. Any available computational methods may be used to build the three dimensional model. As a starting point, the X-ray diffraction pattern obtained from the assemblage of the molecules or atoms in a crystalline version of ACC2 CT or an ACC2 CT homolog can be used to build an electron density map using tools well known to those skilled in the art of crystallography and X-ray diffraction techniques. Additional phase information extracted either from the diffraction data and available in the published literature and/or from supplementing experiments may then used to complete the reconstruction.

For basic concepts and procedures of collecting, analyzing, and utilizing X-ray diffraction data for the construction of electron densities see, for example, Campbell et al., 1984, Biological Spectroscopy, The Benjamin/Cummings Publishing Co., Inc., Menlo Park, Calif.; Cantor et al., 1980, Biophysical Chemistry, Part II: Techniques for the study of biological structure and function, W. H. Freeman and Co., San Francisco, Calif.; A. T. Brunger, 1993, X-Flor Version 3. 1: A system for X-ray crystallography and NMR, Yale Univ. Pr., New Haven, Conn.; M. M. Woolfson, 1997, An Introduction to X-ray Crystallography, Cambridge Univ. Pr., Cambridge, UK; J. Drenth, 1999, Principles of Protein X-ray Crystallography (Springer Advanced Texts in Chemistry), Springer Verlag; Berlin; Tsirelson et al., 1996, Electron Density and Bonding in Crystals: Principles, Theory and X-ray Diffraction Experiments in Solid State Physics and Chemistry, Inst. of Physics Pub.; U.S. Pat. No. 5,942,428; U.S. Pat. No. 6,037,117; U.S. Pat. No. 5,200,910 and U.S. Pat. No. 5,365,456 (“Method for Modeling the Electron Density of a Crystal”), each of which is herein specifically incorporated by reference in their entirety.

For basic information on molecular modeling, see, for example, M. Schlecht, Molecular Modeling on the PC, 1998, John Wiley & Sons; Gans et al., Fundamental Principals of Molecular Modeling, 1996, Plenum Pub. Corp.; N. C. Cohen (editor), Guidebook on Molecular Modeling in Drug Design, 1996, Academic Press; and W. B. Smith, Introduction to Theoretical Organic Chemistry and Molecular Modeling, 1996. U.S. Patents which provide detailed information on molecular modeling include U.S. Pat. Nos. 6,093,573; 6,080,576; 6,075,014; 6,075,123; 6,071,700; 5,994,503; 5,612,894; 5,583,973; 5,030,103; 4,906,122; and 4,812,12, each of which are incorporated by reference herein in their entirety.

### B. Methods of Using the Atomic Coordinates To Identify And Design Ligands of Interest

The atomic coordinates of the invention, such as those described in Table 1, or coordinates substantially identical to or homologous to those of Table 1 may be used with any available methods to prepare three dimensional models of ACC2 CT as well as to identify and design ACC2 CT ligands, inhibitors or antagonists or agonist molecules.

For instance, three-dimensional modeling may be performed using the experimentally determined coordinates derived from X-ray diffraction patterns, such as those in Table 1, for example, wherein such modeling includes, but is not limited to, drawing pictures of the actual structures, building physical models of the actual structures, and determining the structures of related subunits and ACC2 CT/ligand and ACC2 CT subunit/ligand complexes using the coordinates. Such molecular modeling can utilize known X-ray diffraction molecular modeling algorithms or molecular modeling software to generate atomic coordinates corresponding to the three-dimensional structure of ACC2 CT.

As described above, molecular modeling involves the use of computational methods, preferably computer assisted methods, to build realistic models of molecules that are identifiably related in sequence to the known crystal structure. It also involves modeling new small molecule inhibitors bound to ACC2 CT starting with the structures of ACC2 CT and or ACC2 CT complexed with known ligands or inhibitors. The methods utilized in ligand modeling range from molecular graphics (i.e., 3 D representations) to computational chemistry (i.e., calculations of the physical and chemical properties) to make predictions about the binding of ligands or activities of ligands; to design new ligands; and to predict novel molecules, including ligands such as drugs, for chemical synthesis, collectively referred to as rational drug design.

One approach to rational drug design is to search for known molecular structures that might bind to an active site. Using molecular modeling, rational drug design programs can look at a range of different molecular structures of drugs that may fit into the active site of an enzyme, and by moving them in a three-dimensional environment it can be decided which structures actually fit the site well.

An alternative but related rational drug design approach starts with the known structure of a complex with a small molecule ligand and models modifications of that small molecule in an effort to make additional favourable interactions with ACC2 CT.

The present invention include the use of molecular and computer modeling techniques to design and select and design ligands, such as small molecule agonists or antagonists or other therapeutic agents that interact with ACC2 CT. For example, the invention as herein described includes the design of ligands that act as competitive inhibitors of at least one ACC2 CT function by binding to all, or a portion of, the active sites or other regions of ACC2 CT.

This invention also includes the design of compounds that act as uncompetitive inhibitors of at least one function of ACC2 CT. These inhibitors may bind to all, or a portion of, the active sites or other regions of ACC2 CT already bound to its substrate and may be more potent and less non-specific than competitive inhibitors that compete for ACC2 CT active sites. Similarly, non-competitive inhibitors that bind to and inhibit at least one function of ACC2 CT whether or not it is bound to another chemical entity may be designed using the atomic coordinates of ACC2 CT or complexes comprising ACC2 CT of this invention.

The atomic coordinates of the present invention also provide the needed information to probe a crystal of ACC2 CT with molecules composed of a variety of different chemical features to determine optimal sites for interaction between candidate inhibitors and/or activators and ACC2 CT. For example, high resolution X-ray diffraction data collected from crystals saturated with solvent allows the determination of where each type of solvent molecule sticks. Small molecules that bind to those sites can then be designed and synthesized and tested for their inhibitory activity (Travis, J., Science 262: 1374 (1993)).

The present invention also includes methods for computationally screening small molecule databases and libraries for chemical entities, agents, ligands, or compounds that can bind in whole, or in part, to ACC2 CT. In this screening, the quality of fit of such entities or compounds to the binding site or sites may be judged either by shape complementarity or by estimated interaction energy (Meng, E. C. et al., J. Coma. Chem. 13:505-524 (1992)).

The design of compounds that bind to, promote or inhibit the functional activity of ACC2 CT according to this invention generally involves consideration of two factors. First, the compound must be capable of physically and structurally associating with ACC2 CT. Non-covalent molecular interactions important in the association of ACC2 CT with the compound, include hydrogen bonding, van der Waals and hydrophobic interactions. Second, the compound must be able to assume a conformation that allows it to associate with ACC2 CT. Although certain portions of the compound may not directly participate in the association with ACC2 CT, those portions may still influence the overall conformation of the molecule. This, in turn, may have a significant impact on binding affinities, therapeutic efficacy, drug-like qualities and potency. Such conformational requirements include the overall three-dimensional structure and orientation of the chemical entity or compound in relation to all or a portion of the active site or other region of ACC2 CT, or the spacing between functional groups of a compound comprising several chemical entities that directly interact with ACC2 CT.

The potential, predicted, inhibitory agonist, antagonist or binding effect of a ligand or other compound on ACC2 CT may be analyzed prior to its actual synthesis and testing by the use of computer modeling techniques. If the theoretical structure of the given compound suggests insufficient interaction and association between it and ACC2 CT, synthesis and testing of the compound may be obviated. However, if computer modeling indicates a strong interaction, the molecule may then be synthesized and tested for its ability to interact with ACC2 CT. In this manner, synthesis of inoperative compounds may be avoided. In some cases, inactive compounds are synthesized predicted on modeling and then tested to develop a SAR (structure-activity relationship) for compounds interacting with a specific region of ACC2 CT.

One skilled in the art may use one of several methods to screen chemical entities fragments, compounds, or agents for their ability to associate with ACC2 CT and more particularly with the individual binding pockets or active sites of ACC2 CT. This process may begin by visual inspection of, for example, the active site on the computer screen based on the atomic coordinates of ACC2 CT or ACC2 CT complexed with a ligand. Selected chemical entities, compounds, or agents may then be positioned in a variety of orientations, or docked within an individual binding pocket of ACC2 CT. Docking may be accomplished using software such as Quanta and Sybyl, followed by energy minimization and molecular dynamics with standard molecular mechanics forcefields, such as CHARMM and AMBER.

Specialized computer programs may also assist in the process of selecting chemical entities. These include but are not limited to: GRID (Goodford, P. J., “A Computational Procedure for Determining Energetically Favorable Binding Sites on Biologically Important Macromolecules,” J. Med. Chem. 28:849-857 (1985), available from Oxford University, Oxford, UK); MCSS (Miranker, A. and M. Karplus, “Functionality Maps of Binding Sites: A Multiple Copy Simultaneous Search Method.” Proteins: Structure, Function and Genetics 11: 29-34 (1991), available from Molecular Simulations, Burlington, Mass.); AUTODOCK (Goodsell, D. S. and A. J. Olsen, “Automated Docking of Substrates to Proteins by Simulated Annealing” Proteins: Structure. Function, and Genetics 8:195-202 (1990), available from Scripps Research Institute, La Jolla, Calif.); and DOCK (Kuntz, I. D. et al., “A Geometric Approach to Macromolecule-Ligand Interactions,” J.-Mol. Biol. 161:269-288 (1982), available from University of California, San Francisco, Calif.).

The use of software such as GRID, a program that determines probable interaction sites between probes with various functional group characteristics and the macromolecular surface, is used to analyze the surface sites to determine structures of similar inhibiting proteins or compounds. The GRID calculations, with suitable inhibiting groups on molecules (e.g., protonated primary amines) as the probe, are used to identify potential hotspots around accessible positions at suitable energy contour levels. The program DOCK may be used to analyze an active site or ligand binding site and suggest ligands with complementary steric properties.

Once suitable chemical entities, compounds, or agents have been selected, they can be assembled into a single ligand or compound or inhibitor or activator. Assembly may proceed by visual inspection of the relationship of the fragments to each other on the three-dimensional image. This may be followed by manual model building using software such as Quanta or Sybyl.

Useful programs to aid in connecting the individual chemical entities, compounds, or agents include but are not limited to: CAVEAT (Bartlett, P. A. et al., “CAVEAT: A Program to Facilitate the Structure-Derived Design of Biologically Active Molecules.” In Molecular Recognition in Chemical and Biological Problems, Special Pub., Royal Chem. Soc., 78, pp. 82-196 (1989)); 3 D Database systems such as MACCS-3 D (MDL Information Systems, San Leandro, Calif. and Martin, Y. C., “3 D Database Searching in Drug Design”, J. Med. Chem. 35: 2145-2154 (1992); and HOOK (available from Molecular Simulations, Burlington, Mass.).

Several methodologies for searching three-dimensional databases to test pharmacophore hypotheses and select compounds for screening are available. These include the program CAVEAT (Bacon et al., J. Mol. Biol. 225:849-858 (1992)). For instance, CAVEAT uses databases of cyclic compounds which can act as “spacers” to connect any number of chemical fragments already positioned in the active site. This allows one skilled in the art to quickly generate hundreds of possible ways to connect the fragments already known or suspected to be necessary for tight binding.

Instead of proceeding to build an inhibitor activator, agonist or antagonist of ACC2 CT in a step-wise fashion one chemical entity at a time as described above, such compounds may be designed as a whole or “de novo” using either an empty active site or optionally including some portion(s) of a known molecule(s). These methods include: LUDI (Bohm, H.-J., “The Computer Program LUDI: A New Method for the De Novo Design of Enzyme Inhibitors”, J. ComR. Aid. Molec. Design, 6, pp. 61-78 (1992), available from Biosym Technologies, San Diego, Calif.); LEGEND (Nishibata, Y. and A. Itai, Tetrahedron 47:8985 (1991), available from Molecular Simulations, Burlington, Mass.); and LeapFrog (available from Tripos Associates, St. Louis, Mo.).

For instance, the program LUDI can determine a list of interaction sites into which to place both hydrogen bonding and hydrophobic fragments. LUDI then uses a library of linkers to connect up to four different interaction sites into fragments. Then smaller “bridging” groups such as —CH2— and —COO— are used to connect these fragments. For example, for the enzyme DHFR, the placements of key functional groups in the well-known inhibitor methotrexate were reproduced by LUDI. See also, Rotstein and Murcko, J. Med. Chem. 36: 1700-1710 (1992).

Other molecular modeling techniques may also be employed in accordance with this invention. See, e.g., Cohen, N. C. et al., “Molecular Modeling Software and Methods for Medicinal Chemistry, J. Med. Chem. 33:883-894 (1990). See also, Navia, M. A. and M. A. Murcko, “The Use of Structural Information in Drug Design,” Current Opinions in Structural Biology, 2, pp. 202-210 (1992).

Once a compound has been designed or selected by the above methods, the affinity with which that compound may bind or associate with ACC2 CT may be tested and optimized by computational evaluation and/or by testing biological activity after synthesizing the compound. Inhibitors or compounds may interact with the ACC2 CT in more than one conformation that is similar in overall binding energy. In those cases, the deformation energy of binding is taken to be the difference between the energy of the free compound and the average energy of the conformations observed when the compound binds to ACC2 CT.

A compound designed or selected as binding or associating with ACC2 CT may be further computationally optimized so that in its bound state it would preferably lack repulsive electrostatic interaction with ACC2 CT. Such non-complementary (e.g., electrostatic) interactions include repulsive charge-charge, dipole-dipole and charge-dipole interactions. Specifically, the sum of all electrostatic interactions between the inhibitor and ACC2 CT when the inhibitor is bound, preferably make a neutral or favourable contribution to the enthalpy of binding. Weak binding compounds will also be designed by these methods so as to determine SAR.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa., COPYRGT 1992); AMBER, version 4.0 (P. A. Kollman, University of California at San Francisco, COPYRGT 1994); QUANTA/CHARMM (Molecular Simulations, Inc., Burlington, Mass. COPYRGT 1994); and Insight II/Discover (Biosysm Technologies Inc., San Diego, Calif. COPYRGT 1994). Other hardware systems and software packages will be known to those skilled in the art.

Once a compound that associates with ACC2 CT has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation may be avoided. Such substituted chemical compounds may then be analyzed for efficiency of fit to ACC2 CT by the same computer methods described in detail, above.

### C. Use of Homology Structure Modeling To Design Ligands With Modulated Binding Or Activity To ACC2 CT

The present invention includes the use of the atomic coordinates and structures of ACC2 CT and/or ACC2 CT complexed with an inhibitor to design modifications to starting compounds and derivatives thereof that will bind more tightly or interact more specifically to the target enzyme.

The structure of a complex between the ACC2 CT and the starting compound can be used to guide the modification of that compound to produce new compounds that have other desirable properties for applicable industrial and other uses (e.g., as pharmaceuticals), such as chemical stability, solubility or membrane permeability. (Lipinski et al., Adv. Drug Deliv. Rev. 23:3 (1997)).

Binding compounds, agonists, antagonists and such that are known in the art include but are not limited to acetyl-CoA, biotin, and small molecule antagonists. Such compounds can be diffused into or soaked with the stabilized crystals of ACC2 CT to form a complex for collecting X-ray diffraction data. Alternatively, the compounds, known and unknown in the art, can be cocrystallized with ACC2 CT by mixing the compound with ACC2 CT before precipitation.

To produce custom high affinity and very specific compounds, the structure of ACC2 CT can be compared to the structure of a selected non-targeted molecule and a hybrid constructed by changing the structure of residues at the binding site for a ligand for the residues at the same positions of the non-target molecule. The process whereby this modeling is achieved is referred to as homology structure modeling. This is done computationally by removing the side chains from the molecule or target of known structure and replacing them with the side chains of the unknown structure put in sterically plausible positions. In this way it can be understood how the shapes of the active site cavities of the targeted and non-targeted molecules differ. This process, therefore, provides information concerning how a bound ligand can be chemically altered in order to produce compounds that will bind tightly and specifically to the desired target but will simultaneously be sterically prevented from binding to the non-targeted molecule. Likewise, knowledge of portions of the bound ligands that are facing to the solvent would allow introduction of other functional groups for additional pharmaceutical purposes. The use of homology structure modeling to design molecules (ligands) that bind more tightly to the target enzyme than to the non-target enzyme has wide spread applicability.

### D. High Throughput Assays

Any high throughput screening may be utilized to test new compounds which are identified or designed for their ability to interact with ACC2 CT. For general information on high-throughput screening see, for example, Devlin, 1998, High Throughput Screening, Marcel Dekker; and U.S. Pat. No. 5,763,263. High throughput assays utilize one or more different assay techniques including, but not limited to, those described below.

Immunodiagnostics and Immunoassays. These are a group of techniques used for the measurement of specific biochemical substances, commonly at low concentrations in complex mixtures such as biological fluids, that depend upon the specificity and high affinity shown by suitably prepared and selected antibodies for their complementary antigens. A substance to be measured must, of necessity, be antigenic—either an immunogenic macromolecule or a haptenic small molecule. To each sample a known, limited amount of specific antibody is added and the fraction of the antigen combining with it, often expressed as the bound:free ratio, is estimated, using as indicator a form of the antigen labeled with radioisotope (radioimmunoassay), fluorescent molecule (fluoroimmunoassay), stable free radical (spin immunoassay), enzyme (enzyme immunoassay), or other readily distinguishable label.

Antibodies can be labeled in various ways, including: enzyme-linked immunosorbent assay (ELISA); radioimmuno assay (RIA); fluorescent immunoassay (FIA); chemiluminescent immunoassay (CLIA); and labeling the antibody with colloidal gold particles (immunogold).

Common assay formats include:

Enzyme-linked immunosorbent assay (ELISA). ELISA is an immunochemical technique that avoids the hazards of radiochemicals and the expense of fluorescence detection systems. Instead, the assay uses enzymes as indicators. ELISA is a form of quantitative immunoassay based on the use of antibodies (or antigens) that are linked to an insoluble carrier surface, which is then used to “capture” the relevant antigen (or antibody) in the test solution. The antigen-antibody complex is then detected by measuring the activity of an appropriate enzyme that had previously been covalently attached to the antigen (or antibody).

For information on ELISA techniques, see, for example, Crowther, (1995) ELISA—Theory and Practice (Methods in Molecular Biology), Humana Press; Challacombe & Kemeny, (1998) ELISA and Other Solid Phase Immunoassays—Theoretical and Practical Aspects, John Wiley; Kemeny, (1991) A Practical Guide to ELISA, Pergamon Press; Ishikawa, (1991) Ultrasensitive and Rapid Enzyme Immunoassay (Laboratory Techniques in Biochemistry and Molecular Biology) Elsevier.

Colorimetric Assays for Enzymes. Colorimetry is any method of quantitative chemical analysis in which the concentration or amount of a compound is determined by comparing the color produced by the reaction of a reagent with both standard and test amounts of the compound, often using a calorimeter. A calorimeter is a device for measuring color intensity or differences in color intensity, either visually or photoelectrically.

Standard calorimetric assays of beta-galactosidase enzymatic activity are well known to those skilled in the art (see, for example, Norton et al., Mol. Cell. Biol. 5:281-290 (1985). A calorimetric assay can be performed on whole cell lysates using O-nitrophenyl-beta-D-galacto-pyranoside (ONPG, Sigma) as the substrate in a standard calorimetric beta-galactosidase assay (Sambrook et al., (1989) Molecular Cloning—A Laboratory Manual, Cold Spring Harbor Laboratory Press). Automated calorimetric assays are also available for the detection of beta-galactosidase activity, as described in U.S. Pat. No. 5,733,720.

### E. Databases And Computer Systems

An amino acid sequence or nucleotide sequence of ACC2 CT and/or X-ray diffraction data, useful for computer molecular modeling of ACC2 CT or a portion thereof, can be “provided” in a variety of mediums to facilitate use thereof. As used herein, “provided” refers to a manufacture, which contains, for example, an amino acid sequence or nucleotide sequence and/or atomic coordinates derived from X-ray diffraction data of the present invention, e.g., an amino acid or nucleotide sequence of ACC2 CT, a representative fragment thereof, or a homologue thereof. Such a method provides the amino acid sequence and/or X-ray diffraction data in a form which allows a skilled artisan to analyze and molecular model the three-dimensional structure of ACC2 CT or related molecules, including a subdomain thereof.

In one application of this embodiment, databases comprising data pertaining to ACC2 CT, or at least one subdomain thereof, amino acid and nucleic acid sequence and/or X-ray diffraction data of the present invention is recorded on computer readable medium. As used herein, “computer readable medium” refers to any medium which can be read and accessed directly by a computer. Such media include, but are not limited to: magnetic storage media, such as floppy discs, hard disc storage media, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon an amino acid sequence and/or X-ray diffraction data of the present invention.

As used herein, “recorded” refers to a process for storing information on computer readable media. A skilled artisan can readily adopt any of the presently known methods for recording information on computer readable media to generate manufactures comprising an amino acid sequence and/or atomic coordinate/X-ray diffraction data information of the present invention.

A variety of data storage structures are available to a skilled artisan for creating a computer readable medium having recorded thereon an amino acid sequence and/or atomic coordinate/X-ray diffraction data of the present invention. The choice of the data storage structure will generally be based on the means chosen to access the stored information. In addition, a variety of data processor programs and formats can be used to store the sequence and X-ray data information of the present invention on computer readable media. The sequence information can be represented in a word processing text file, formatted in commercially-available software such as WordPerfect and MICROSOFT Word, or represented in the form of an ASCII file, stored in a database application, such as DB2, Sybase, Oracle, or the like. A skilled artisan can readily adapt any number of dataprocessor structuring formats (e.g., text file or database) in order to obtain computer readable media having recorded thereon the information of the present invention.

By providing computer readable media having sequence and/or atomic coordinates based on X-ray diffraction data, a skilled artisan can routinely access the sequence and atomic coordinate or X-ray diffraction data to model a related molecule, a subdomain, mimetic, or a ligand thereof. Computer algorithms are publicly and commercially available which allow a skilled artisan to access this data provided in a computer readable medium and analyze it for molecular modeling and/or RDD (rational drug design). See, e.g., Biotechnology Software Directory, MaryAnn Liebert Publ., New York (1995).

The present invention further provides systems, particularly computer-based systems, which contain the sequence and/or diffraction data described herein. Such systems are designed to do structure determination and RDD for ACC2 CT or at least one subdomain thereof. Non-limiting examples are microcomputer workstations available from Silicon Graphics Incorporated and Sun Microsystems running UNIX based, Windows NT or IBM OS/2 operating systems.

As used herein, “a computer-based system” refers to the hardware means, software means, and data storage means used to analyze the sequence and/or X-ray diffraction data of the present invention. The minimum hardware means of the computer-based systems of the present invention comprises a central processing unit (CPU), input means, output means, and data storage means. A skilled artisan can readily appreciate which of the currently available computer-based systems are suitable for use in the present invention. A visualization device, such as a monitor, is optionally provided to visualize structure data.

As stated above, the computer-based systems of the present invention comprise a data storage means having stored therein sequence and/or atomic coordinate/X-ray diffraction data of the present invention and the necessary hardware means and software means for supporting and implementing an analysis means. As used herein, “data storage means” refers to memory which can store sequence or atomic coordinate/X-ray diffraction data of the present invention, or a memory access means which can access manufactures having recorded thereon the sequence or X-ray data of the present invention.

As used herein, “search means” or “analysis means” refers to one or more programs which are implemented on the computer-based system to compare a target sequence or target structural motif with the sequence or X-ray data stored within the data storage means. Search means are used to identify fragments or regions of a protein which match a particular target sequence or target motif. A variety of known algorithms are disclosed publicly and a variety of commercially available software for conducting search means are and can be used in the computer-based systems of the present invention. A skilled artisan can readily recognize that any one of the available algorithms or implementing software packages for conducting computer analyses can be adapted for use in the present computer-based systems.

As used herein, “a target structural motif,” or “target motif,” refers to any rationally selected sequence or combination of sequences in which the sequence(s) are chosen based on a three-dimensional configuration or electron density map which is formed upon the folding of the target motif. There are a variety of target motifs known in the art. Protein target motifs include, but are not limited to, enzymatic active sites, inhibitor binding sites, structural subdomains, epitopes, functional domains and signal sequences. Similar motifs are known for RNA. A variety of structural formats for the input and output means can be used to input and output the information in the computer-based systems of the present invention.

A variety of comparing means can be used to compare a target sequence or target motif with the data storage means to identify structural motifs or electron density maps derived in part from the atomic coordinate/X-ray diffraction data. A skilled artisan can readily recognize that any one of the publicly available computer modeling programs can be used as the search means for the computer-based systems of the present invention.

### F. Target Molecule Fragments And Portions

Fragments of ACC2 CT, for instance fragments comprising active sites defined by two or more amino acids selected from the group consisting of: A459-A462, A530-A538, B261-B270 may be prepared by any available means including synthetic or recombinant means. Such fragments may then be used in the assays as described above, for instance, high throughput assays to detect interactions between prospective agents and the active site within the fragment.

For recombinant expression or production of the fragments of the invention, nucleic acid molecules encoding the fragment may be prepared. As used herein, “nucleic acid” is defined as RNA or DNA that encodes a protein or peptide as defined above, or is complementary to nucleic acid sequence encoding such peptides, or hybridizes to such nucleic acid and remains stably bound to it under appropriate stringency conditions.

Nucleic acid molecules encoding fragments of the invention may differ in sequence because of the degeneracy in the genetic code or may differ in sequence as they encode proteins or protein fragments that differ in amino acid sequence. Homology or sequence identity between two or more such nucleic acid molecules is determined by BLAST (Basic Local Alignment Search Tool) analysis using the algorithm employed by the programs blastp, blastn, blastx, tblastn and tblastx (Karlin et al., Proc. Natl. Acad. Sci. USA 87:2264-2268 (1990) and Altschul, et al., J. Mol. Evol. 36:290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nat. Genet. 6, 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89:10915-10919 (1992), fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C. or (2) employ during hybridization a denaturing agent such as formamide, for example, 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC, 50 mM sodium phosphate (pH 6.8), 0.1% sodium pyrophosphate, 5× Denhardt's solution, sonicated salmon sperm DNA (50 mg/ml), 0.1% SDS and 10% dextran sulfate at 42° C., with washes at 42° C. in 0.2×SSC and 0.1% SDS. A skilled artisan can readily determine and vary the stringency conditions appropriately to obtain a clear and detectable hybridization signal.

As used herein, a nucleic acid molecule is said to be “isolated” when the nucleic acid molecule is substantially separated from contaminant nucleic acid encoding other polypeptides from the source of nucleic acid.

The encoding nucleic acid molecules of the present invention (i.e., synthetic oligonucleotides) and those that are used as probes or specific primers for polymerase chain reaction (PCR) or to synthesize gene sequences encoding proteins of the invention can easily be synthesized by chemical techniques, for example, the phosphotriester method of Matteucci et al. (J. Am. Chem. Soc. 103: 185-3191 (1981)) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art-known labels to obtain a labeled encoding nucleic acid molecule.

The present invention further provides recombinant DNA molecules (rDNA) that contain a coding sequence for a protein fragment as described above. As used herein, a rDNA molecule is a DNA molecule that has been subjected to molecular manipulation. Methods for generating rDNA molecules are well known in the art, for example, see Sambrook et al. Molecular Cloning—A Laboratory Manual, Cold Spring Harbor Laboratory Press (1989). In the preferred rDNA molecules, a coding DNA sequence is operably linked to expression control sequences and/or vector sequences.

The choice of vector and expression control sequences to which one of the protein encoding sequences of the present invention is operably linked depends directly, as is well known in the art, on the functional properties desired (e.g., protein expression, and the host cell to be transformed). A vector of the present invention may be capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

The present invention further provides host cells transformed with a nucleic acid molecule that encodes a protein fragment of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, insect, yeast, and mammalian cells. Preferred eukaryotic host cells include Sf9 insect cells.

Transformed host cells of the invention may be cultured under conditions that allow the production of the recombinant protein. Optionally the recombinant protein is isolated from the medium or from the cells; recovery and purification of the protein may not be necessary in some instances where some impurities may be tolerated.

Kits may also be prepared with any of the above described nucleic acid molecules, protein fragments, vector and/or host cells optionally packaged with the reagents needed for a specific assay, such as those described above. In such kits, the protein fragments or other reagents may be attached to a solid support, such as glass or plastic beads.

### G. Integrated Procedures Which Utilize the Present Invention

Molecular modeling is provided by the present invention for rational drug design (RDD) of mimetics and ligands of ACC2 CT. As described above, the drug design paradigm uses computer modeling programs to determine potential mimetics and ligands which are expected to interact with sites on the protein. The potential mimetics or ligands are then screened for activity and/or binding and/or interaction. For ACC2 CT-related mimetics or ligands, screening methods can be selected from assays for at least one biological activity of ACC2 CT, e.g., such as decreased production of malonyl-CoA in muscle tissue. See, for example, Harwood et al., J. Biol. Chem., Vol. 278, Issue 39, 37099-37111, Sep. 26, 2003.

Thus, the tools and methodologies provided by the present invention may be used in procedures for identifying and designing ligands which bind in desirable ways with the target. Such procedures utilize an iterative process whereby ligands are synthesized, tested and characterized. New ligands can be designed based on the information gained in the testing and characterization of the initial ligands and then such newly identified ligands can themselves be tested and characterized. This series of processes may be repeated as many times as necessary to obtain ligands with the desirable binding properties.

The following steps (1-7) serve as an example of the overall procedure:

1.) A biological activity of a target is selected (e.g., production of malonyl-CoA by actylCoA carboxylase).

2.) A ligand is identified that appears to be in some way associated with the chosen biological activity (e.g., the ligand may be an inhibitor of a known activity). The activity of the ligand may be tested by in vivo and/or in vitro methods.

A ligand of the present invention can be, but is not limited to, at least one selected from a lipid, a nucleic acid, a compound, a protein, an element, an antibody, a saccharide, an isotope, a carbohydrate, an imaging agent, a lipoprotein, a glycoprotein, an enzyme, a detectable probe, and antibody or fragment thereof, or any combination thereof, which can be detectably labeled as for labeling antibodies. Such labels include, but are not limited to, enzymatic labels, radioisotope or radioactive compounds or elements, fluorescent compounds or metals, chemiluminescent compounds and bioluminescent compounds. Alternatively, any other known diagnostic or therapeutic agent can be used in a method of the invention. Suitable compounds are then tested for activities in relationship to the target.

Complexes between ACC2 CT and ligands are made either by co-crystallization or more commonly by diffusing the small molecule ligand into the crystal. X-ray diffraction data from the complex crystal are measured and a difference electron density map is calculated. This process provides the precise location of the bound ligand on the target molecule. The difference Fourier is calculated using measure diffraction amplitudes and the phases of these reflections calculated from the coordinates.

3.) Using the methods of the present invention, X-ray crystallography is utilized to create electron density maps and/or molecular models of the interaction of the ligand with the target molecule.

The entry of the coordinates of the target into the computer programs discussed above results in the calculation of most probable structure of the macromolecule. These structures are combined and refined by additional calculations using such programs to determine the probable or actual three-dimensional structure of the target including potential or actual active or binding sites of ligands. Such molecular modeling (and related) programs useful for rational drug design of ligands or mimetics, are also provided by the present invention.

4.) The electron density maps and/or molecular models obtained in Step 3 are compared to the electron density maps and/or molecular models of a non-ligand containing target and the observed/calculated differences are used to specifically locate the binding of the ligand on the target or subunit.

5.) Modeling tools, such as computational chemistry and computer modeling, are used to adjust or modify the structure of the ligand so that it can make additional or different interactions with the target.

The ligand design uses computer modeling programs which calculate how different molecules interact with the various sites of the target, subunit, or a fragment thereof. Thus, this procedure determines potential ligands or ligand mimetics.

6.) The newly designed ligand from Step 5 can be tested for its biological activity using appropriate in vivo or in vitro tests, including the high throughput screening methods discussed above.

The potential ligands or mimetics are then screened for activity relating to ACC2 CT, or at least a fragment thereof. Such screening methods are selected from assays for at least one biological activity of the native target.

The resulting ligands or mimetics, provided by methods of the present invention, are useful for treating, screening or preventing diseases in animals, such as mammals (including humans).

7.) Of course, each of the above steps can be modified as desired by those of skill in the art so as to refine the procedure for the particular goal in mind. Also, additional X-ray diffraction data may be collected on ACC2 CT, ACC2 CT/ligand complexes, ACC2 CT structural target motifs and ACC2 CT subunit/ligand complexes at any step or phase of the procedure. Such additional diffraction data can be used to reconstruct electron density maps and molecular models, which may further assist in the design and selection of ligands with the desirable binding attributes.

It is to be understood that the present invention is considered to include stereoisomers as well as optical isomers, e.g., mixtures of enantiomers as well as individual enantiomers and diastereomers, which arise as a consequence of structural asymmetry in selected compounds, ligands or mimetics of the present series.

Some of the compounds or agents disclosed or discovered by the methods herein may contain one or more asymmetric centers and thus give rise to enantiomers, diastereomers, and other stereoisomeric forms. The present invention is also meant to encompass all such possible forms as well as their racemic and resolved forms and mixtures thereof. When the compounds described or discovered herein contain olefinic double bonds or other centers of geometric asymmetry, and unless otherwise specified, it is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

As used herein, the term “chiral center” refers to to a carbon atom to which four different groups are attached.

As used herein, the term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction.

As used herein, the term “racemic” refers to a mixture of equal parts of enantiomers and which is optically active.

As used herein, the term “resolution” refers to the separation or concentration or depletion of one of the two enantiomeric forms of a molecule. In the context of this application. the term “resolution” also refers to the amount of detail which can be resolved by the diffraction experiment. Or in other terms, since the inherent disorder of a protein crystal diffraction pattern fades away at some diffraction angle thetaₘₐₓ, the corresponding distance dₘᵢₙ of the reciprocal lattices is determined by Bragg's law. 1 d mm=2 sin max

In practice in protein crystallography it is usual to quote the nominal resolution of a protein electron density in terms of dₘᵢₙ, the minimum lattice distance to which data is included in the calculation of the map.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### H. NADH Reverse-Coupled Assay

The NADH reverse-coupled assay was used to measure specific activity of different carboxyl transferase domain constructs of human actyl-CoA carboxylase 2 and human actyl-CoA carboxylase 1. (Guchhait et al., 1974.) It was also used to calculate % inhibition values for selected inhibitors.

Literature suggests a c-terminal fragment consisting of just the CT domain has activity comparable to the full-length enzyme, although the activity only represents the second half-reaction of the full-length enzyme. (Jelenska et al., 2002) The activity for the second half-reaction can be measured in the reverse direction, by quantifying the amount of acetyl-CoA generated from decarboxylation of malonyl-CoA. (Guchhait et al., 1974.) The decarboxylation reaction can proceed by biotin-dependant as well as biotin-independent mechanisms and it has been demonstrated that inhibition of the biotin-dependent component of the reverse reaction is comparable to inhibition of the full reaction for the full-length enzyme. (Jelenska et al., 2002)

In the reverse reaction, ACC2 CT catalyzes the production of Acetyl CoA using malonyl CoA and biocytin as the substrates. Note that biotin is the native substrate and it is covalently bound to the BCC domain of the full-length enzyme. Biocytin, which is biotin bound to lysine, is used in the reverse enzyme assay because it is more soluble than biotin and because it was demonstrated to be a better substrate. (Polakis et al., 1974.)

The activity of ACC2 CT in the reverse reaction is measured indirectly by coupling the reaction with two other enzymes, malate dehydrogenase and citrate synthase. Malate dehydrogenase converts NAD+ and malate to produce NADH and oxaloacetate. The acetyl CoA produced by the ACC2 CT reverse reaction and the oxaloacetate produced by the malate dehydrogenase reaction are consumed as the substrates for the citrate synthase reaction. The final products of the citrate synthase reaction are citric acid and CoA, but it is the production of NADH that acts as the readout for the activity of ACC2 CT. The conversion of NAD+ to NADH is detected by reading absorbance at 340 nm.

The final assay conditions are 50 nM ACC2 CT, 1 mM Malonyl CoA, 20 mM Biocytin, 8 mM Malic Acid, 3 mM NAD+, 100 units/mL Malate Dehydrogenase, 20 units/mL Citrate Synthase, 50 mM Hepes pH 7.0, 100 mM NaCl, 0.01% Tween 20, and compounds are used with a 200-fold dilution of 100% DMSO stock for a final concentration of 0.5% DMSO.

Absorbance data is collected at 340 nm and 37° C. for at least 30 minutes. Linear kinetic rates (mOD/min) are used to calculate % inhibition values of the compounds tested. The kM of Malonyl CoA against 50 nM ACC2/20 mM Biocytin is 160 uM. The kM of Malonyl CoA against ACC2/No Biocytin is 130 uM. The kM of Biocytin could not be precisely determined, but it was estimated to be ˜20 mM.

### I. ExSAR's Proprietary H/D-Ex Platform

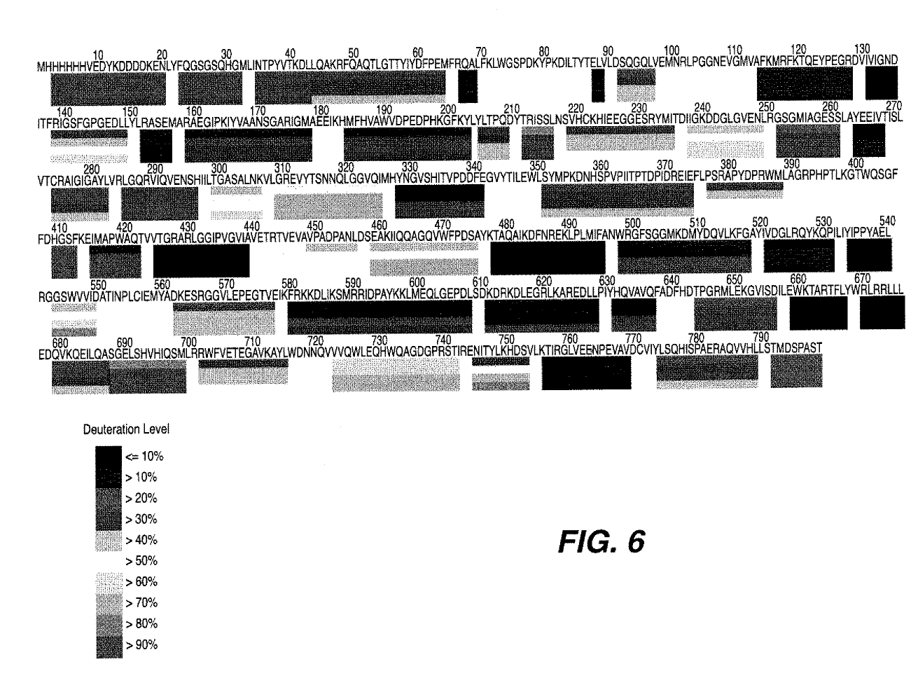

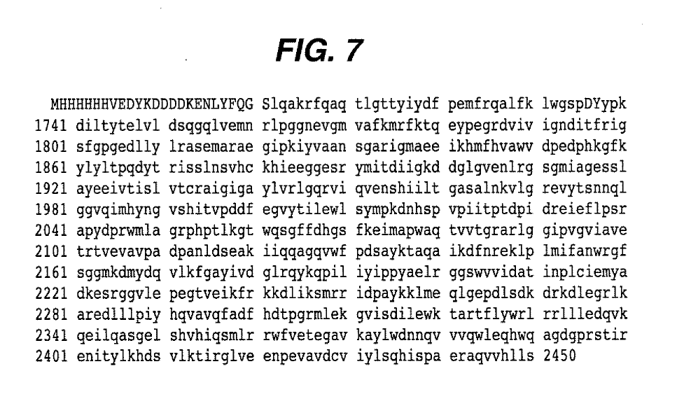

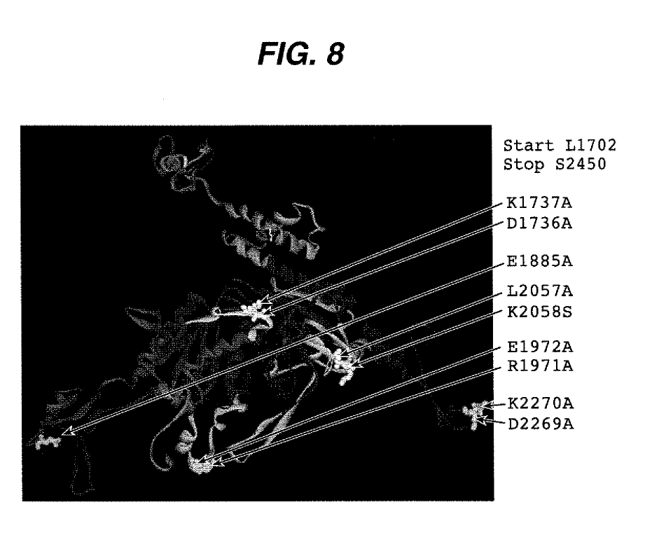

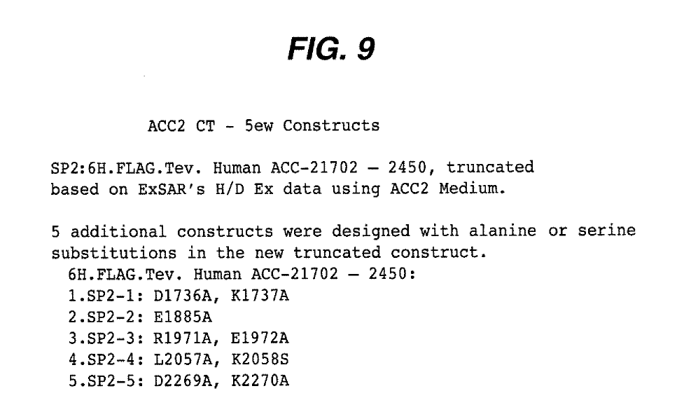

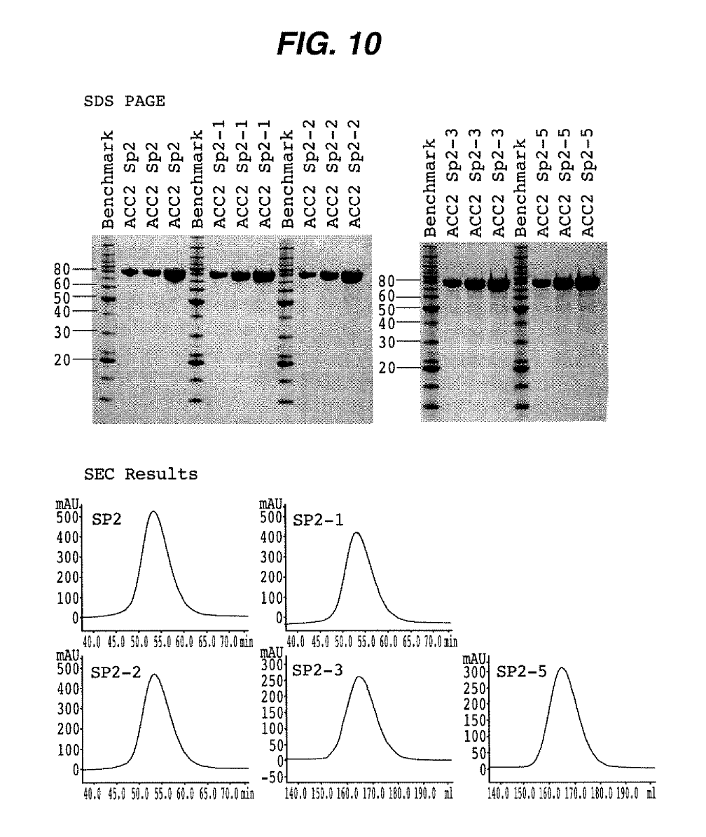

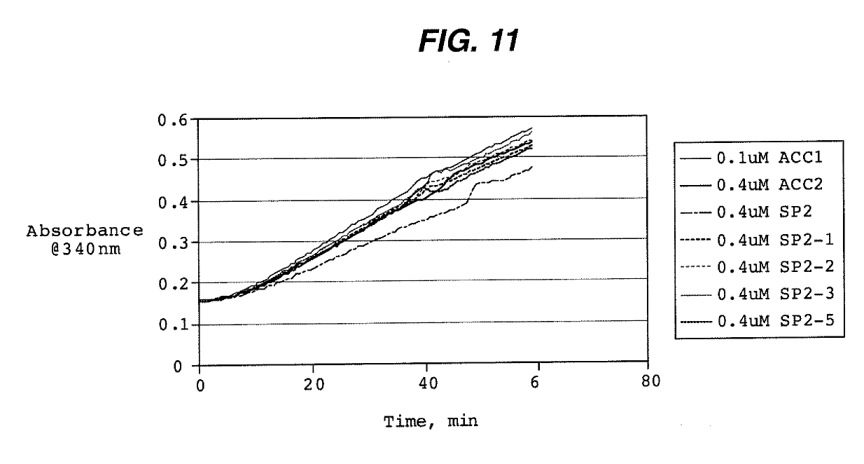

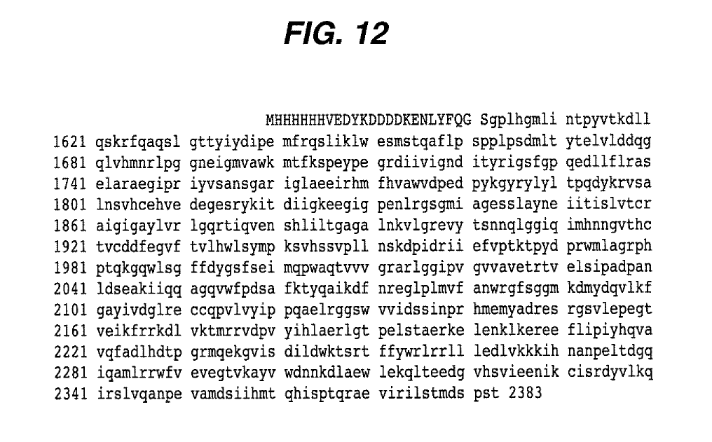

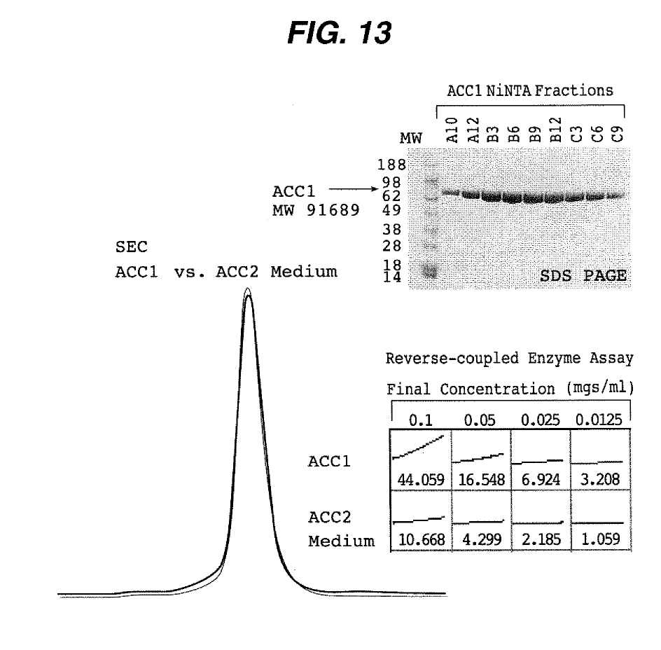

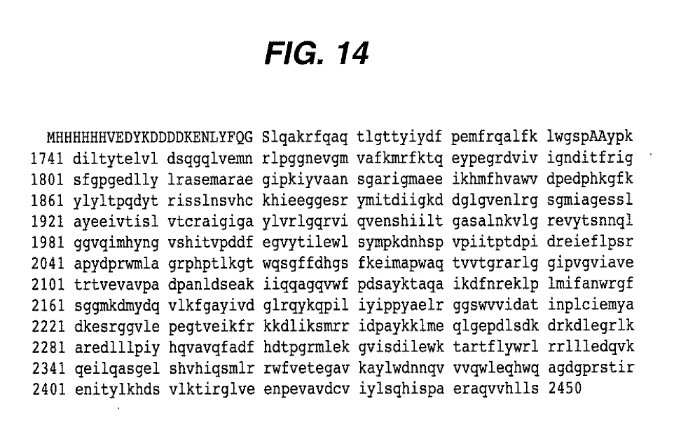

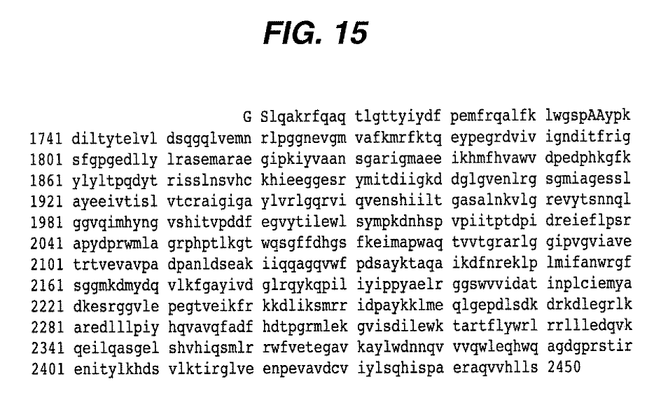

ExSAR's proprietary H/D-Ex platform was used to determine the location of flexible regions in the ACC2 Medium construct. For deuterium labeling, a sample of 5 uL of 2.1 mg/ml (23.9 uM) ACC2 Medium was mixed with 15 uL D2O in 25 mM HEPES buffer, pH 7.0. The reaction solution was incubated at 4° C. for predetermined duration times of 15, 50, 150, and 500 seconds. The reaction was quenched by mixing it with 30 uL of low pH and low temperature solution. The quenched reaction was injected into ExSAR's H/D-Ex system. A fully deuterated sample was made to the same on-exchange concentration by adding 20 uL of the protein sample to 60 uL of 100 mM TCEP in D2O, and incubating at 60° C. overnight. Various conditions were tried for optimization of the protease digestion of the protein. The variables included; type of protease column, type and concentration of denaturants in the quenching buffer, type and concentration of acid in the quenching buffer, and digestion time as determined by flow rate over the protease column. RP-HPLC separation conditions were also optimized. The optimized conditions were pepsin and a quench buffer of 6.4 M GuHCl and 0.8% formic acid, a flow rate over the pepsin column of 200 uL/min, and HPLC gradient that was 12% acetonitrile to 38% acetonitrile in 23 min. The final coverage for the ACC2 Medium protein was 95% (=758/799 amino acids). The H/D-Ex Profile of ACC2 Medium is shown in FIG. 6. The high-resolution structural data shows a large flexible region at the N-terminus and a small flexible portion at the C-terminus of the ACC2 Medium protein.

### J. ACC-2 (SP2-1) Cloning

Human ACC2 (1702-2450.D1736A.K1737A) gene was synthesized and subcloned into pENTR11 vector. Transfection-grade DNA was purified using the QIAwell Kit from Qiagen. LR reaction was performed overnight and then transfected into Sf9 cells using BaculoDirect Baculovirus Expression System (Invitrogen). P0 virus was collected 4 days post transfection and used for another round of virus amplification. P1 virus and cells were collected 3 days post-infection.

P2 virus was expanded to generate a high titer P3 stock for recombinant protein expression by infecting Sf9 cells in suspension at an MOI of 0.3 and harvesting the virus after 72 hours. Cell paste for protein purification was obtained by infecting Sf9 cells at a density of 1.5 e⁶/ml with an MOI of 1. Cultures were maintained at 27 C for 65-72 hours shaking at 140 rpms. Cells were harvested by centrifugation at 1000×g for 10 minutes at 4° C. Following collection, cell pellets were washed in PBS with broad range protease inhibitors and stored at −80° C. Samples were saved for SDS-PAGE and Western blot analysis.

### K. Human ACC2 CT Homology Model

The human ACC2 CT homology model was generated using the ACC2 Medium sequence. A BLAST search of the sequence was performed against the PDBAA (database of publicly accessible protein crystal and NMR structures) to identify appropriate model templates. The crystal structure (B and C chains) from yeast (Saccharomyces Cerevisia—pdb accession 1w2x) was found to have high homology to the human sequence and was subsequently chosen as the model template. Initially, an alignment of the human and yeast sequences was performed using a modified CLUSTALW algorithm of GeneMine's LOOK™ application. The highest scoring alignment, according to the BLOSUM similarity matrix, was used for the model. Next, SEGMOD (LOOK™ suite of applications) created rough Cartesian coordinates which were then subject to stereochemical refinement using a proprietary force-field with 500 cycles of energy minimization. This was performed using both B and C chains from the crystal structure to generate the final human dimer model.

### L. Purification of Human ACC2 1702-2450 (D1736A K1737A)

Frozen cells were thawed and resuspended in 50 mM Tris buffer pH 8.0 containing 400 mM NaCl, 5% glycerol, 0.05% BME, 20 mM imidazole, 2.5 U/ml benzonase, 1 kU/ml rLysozyme, 2× complete EDTA-free protease inhibitor cocktail (Roche). Resuspended cells were dounce homogenized and mechanically lysed with a microfluidizer processor (Microfluidics) at 18,000 psi. The lysate was clarified by centrifugation at 43,000 g for 1 hour. All following purification steps were performed on an ÄKTAxpress system (GE Healthcare) at 4° C. and were fully automated. The supernatant was loaded onto a 1 ml HiTrap crude column (GE Healthcare) and the resin was washed with 30 column volumes of buffer A (50 mM Tris buffer pH 8.0, 400 mM NaCl, 5% glycerol, 0.05% BME, 20 mM imidazole). On column cleavage of the histidine tag was performed by injecting 96 ug of TEV S219V protease/mg of ACC2 CT, and incubating at 4° C. for 20 hours. Cleaved ACC2 was eluted in buffer A and loaded directly onto a HiLoad 16/60 Superdex 200 column (GE Healthcare), preequilibrated with 25 mM Tris Buffer pH 8.0, 200 mM NaCl, 5% Glycerol, 5 mM DTT. Fractions containing ACC2 CT, as assayed by SDS-PAGE, were pooled. Compounds were added in a 1:2 molar ratio of protein versus compound and incubated overnight at 4° C. The various protein:ligand complexes were concentrated to a final protein concentration of 7 mg/ml using an Ultrafree membrane (30 kDa cut-off) and were then ready for crystallization.

### M. Crystallization And Data Collection

A 7 mg/ml protein:ligand complex of TEV-cleaved 6H.FLAG.Tev. Human ACC-2 1702-2450 (D1736A, K1737A) in 25 mM Tris pH 8.0, 200 mM NaCl, 5% Glyercol, 5 mM DTT and the structure in FIG. 16 was used for high throughput crystallization screening (HTXS). Numerous screens were conducted using the HTXS_{—}96well_Index crystallization screen at 22° C.

A single bi-pyramid crystal was generated after 2 months from the HTXS_{—}96well_Index crystallization screen and transferred into a 20% glycerol cryo-protectant. The crystal was subsequently screened for diffraction at Argonne National Laboratory's Advanced Photon Source (APS) 17-ID beamline. Initial diffraction was observed at 5.5 Å.

The same APS crystal was used for seeding experiments. Seeds were produced with a Seed Bead Kit (Hampton Research) by vortexing the crystal in 60 ul of stabilization buffer consisting of 12% PEG 3350; 100 mM Hepes pH 7.5; 200 mM Proline. Protein drops consisted of 1 ul protein solution, 1 ul of well solution, and 0.2 ul of seed solution. The protein drop was suspended over a range of 6% to 9% PEG 3350 in 100 mM Hepes pH 7.5; 200 mM Proline. The experiments generated numerous bi-pyramid crystals. Crystals suitable for X-ray analysis were regenerated within three days and screened at the APS with 3.2 Å diffraction observed. A dataset was collected on April 20, 2006 from ACC2 crystallization tray A041106_{—}4 leading to structure determination of human ACC2 CT.

Lengthy table referenced here
US20090155815A1-20090618-T00001
Please refer to the end of the specification for access instructions.

### REFERENCES

### Patents And Patent Publications

U.S. patent application Ser. No. 10/754,687, which claims the benefit of the priority of the following four US Provisional Applications: U.S. Ser. No. 60/439,383, filed Jan. 10, 2003; 60/459,464, filed Mar. 31, 2003; 60/491, 640, filed Jul. 31, 2003; and 60/514,636, filed Oct. 27, 2003.
U.S. Pat. No. 6,979,741
U.S. Pat. No. 5,942,428; U.S. Pat. No. 6,037,117; U.S. Pat. No. 5,200,910 and U.S. Pat. No. 5,365,456 (“Method for Modeling the Electron Density of a Crystal”).
Patents which provide detailed information on molecular modeling include:
U.S. Pat. Nos. 6,093,573; 6,080,576; 6,075,014; 6,075,123; 6,071,700; 5,994,503; 5,612,894; 5,583,973; 5,030,103; 4,906,122; and 4,812,12.
U.S. Pat. No. 5,763,263.
U.S. Pat. No. 5,733,720.

### Other References

Abu-Elheiga, L., A. Jayakumar, et al. (1995). “Human acetyl-CoA carboxylase: characterization, molecular cloning, and evidence for two isoforms.” Proc Natl Acad Sci USA 92 (9): 4011-5.
Abu-Elheiga, L., D. B. Almarza-Ortega, et al. (1997). “Human acetyl-CoA carboxylase 2. Molecular cloning, characterization, chromosomal mapping, and evidence for two isoforms.” J Biol Chem 272 (16): 10669-77.
Abu-Elheiga, L., M. M. Matzuk, et al. (2001). “Continuous fatty acid oxidation and reduced fat storage in mice lacking acetyl-CoA carboxylase 2.” Science 291 (5513): 2613-6.
Abu-Elheiga, L., W. Oh, et al. (2003). “Acetyl-CoA carboxylase 2 mutant mice are protected against obesity and diabetes induced by high-fat/high-carbohydrate diets.” Proc Natl Acad Sci USA 100 (18): 10207-12.
Abu-Elheiga, L., W. R. Brinkley, et al. (2000). “The subcellular localization of acetyl-CoA carboxylase 2.” Proc Natl Acad Sci USA 97 (4): 1444-9.
Altschul et al. (Nat. Genet. 6, 119-129 (1994)).
Altschul, et al., J. Mol. Evol. 36:290-300 (1993).
Brunger, A. T., 1993, X-Flor Version 3. 1: A system for X-ray crystallography and NMR, Yale Univ. Pr., New Haven, Conn.
Campbell et al., 1984, Biological Spectroscopy, The Benjamin/Cummings Publishing Co., Inc., Menlo Park, Calif.
Cantor et al., 1980, Biophysical Chemistry, Part II: Techniques for the study of biological structure and function, W. H. Freeman and Co., San Francisco, Calif.
Challacombe & Kemeny, (1998) ELISA and Other Solid Phase Immunoassays—Theoretical and Practical Aspects, John Wiley.
Cohen, N. C. (editor), Guidebook on Molecular Modeling in Drug Design, 1996, Academic Press.
Cohen, N. C. et al., “Molecular Modeling Software and Methods for Medicinal Chemistry, J. Med. Chem. 33:883-894 (1990).
Crowther, (1995) ELISA—Theory and Practice (Methods in Molecular Biology), Humana Press.
Derewenda, Z. S. (2004). “Rational protein crystallization by mutational surface engineering.” Structure 12 (4): 529-35.
Devlin, 1998, High Throughput Screening, Marcel Dekker.
Drenth, J., 1999, Principles of Protein X-ray Crystallography (Springer Advanced Texts in Chemistry), Springer Verlag; Berlin.
Gans et al., Fundamental Principals of Molecular Modeling, 1996, Plenum Pub. Corp.
Guchhait, R. B., S. E. Polakis, et al. (1974). “Acetyl coenzyme A carboxylase system of Escherichia coli. Purification and properties of the biotin carboxylase, carboxyltransferase, and carboxyl carrier protein components.” J Biol Chem 249 (20): 6633-45.
Guchhait, R. B., S. E. Polakis, et al. (1975). “Carboxyltransferase component of acetyl-CoA carboxylase from Escherichia coli.” Methods Enzymol 35: 32-7.
Harwood, H. J., Jr. (2004). “Acetyl-CoA carboxylase inhibition for the treatment of metabolic syndrome.” Curr Opin Investig Drugs 5 (3): 283-9.
Harwood, H. J., Jr. (2005). “Treating the metabolic syndrome: acetyl-CoA carboxylase inhibition.” Expert Opin Ther Targets 9 (2): 267-81.
Harwood, H. J., Jr., S. F. Petras, et al. (2003). “Isozyme-nonselective N-substituted bipiperidylcarboxamide acetyl-CoA carboxylase inhibitors reduce tissue malonyl-CoA concentrations, inhibit fatty acid synthesis, and increase fatty acid oxidation in cultured cells and in experimental animals.” J Biol Chem 278 (39): 37099-111.
Henikoff et al., Proc. Natl. Acad. Sci. USA 89:10915-10919 (1992).
Ishikawa, (1991) Ultrasensitive and Rapid Enzyme Immunoassay (Laboratory Techniques in Biochemistry and Molecular Biology) Elsevier.
Karlin et al., Proc. Natl. Acad. Sci. USA 87:2264-2268 (1990).
Kemeny, (1991) A Practical Guide to ELISA, Pergamon Press.
Lipinski et al., Adv. Drug Deliv. Rev. 23:3 (1997).
Matteucci et al. (J. Am. Chem. Soc. 103: 185-3191 (1981)).
Meng, E. C. et al., J. Comp. Chem. 13:505-524 (1992).
Navia, M. A. and M. A. Murcko, “The Use of Structural Information in Drug Design,” Current Opinions in Structural Biology, 2, pp. 202-210 (1992).
Norton et al., Mol. Cell. Biol. 5:281-290 (1985).
Polakis, S. E., R. B. Guchhait, et al. (1974). “Acetyl coenzyme A carboxylase system of Escherichia coli. Studies on the mechanisms of the biotin carboxylase- and carboxyltransferase-catalyzed reactions.” J Biol Chem 249 (20): 6657-67.
Rossmann, M. G. (1972). The Molecular Replacement Method, Gordon & Breach, New York.
Rossmann, M. G., (editor), “The Molecular Replacement Method”, Gordon & Breach, New York, 1972.
Rotstein and Murcko, J. Med. Chem. 36: 1700-1710 (1992).
Sambrook et al. Molecular Cloning-A Laboratory Manual, Cold Spring Harbor Laboratory Press (1989).
Sambrook et al., (1989) Molecular Cloning—A Laboratory Manual, Cold Spring Harbor Laboratory Press.
Schlecht, M., Molecular Modeling on the PC, 1998, John Wiley & Sons.
Segel, I. H. (1975). Enzyme Kinetics, J. Willey & Sons.
Segel, I. H., Enzyme Kinetics, J. Willey & Sons, (1975).
Smith, W. B., Introduction to Theoretical Organic Chemistry and Molecular Modeling, 1996.
Tong, L. (2005). “Acetyl-coenzyme A carboxylase: crucial metabolic enzyme and attractive target for drug discovery.” Cell Mol Life Sci 62(16): 1784-803.
Tong, L. and H. J. Harwood, Jr. (2006). “Acetyl-coenzyme A carboxylases: versatile targets for drug discovery.” J Cell Biochem 99(6): 1476-88.
Travis, J., Science 262:1374(1993).
Tsirelson et al., 1996, Electron Density and Bonding in Crystals: Principles, Theory and X-ray Diffraction Experiments in Solid State Physics and Chemistry, Inst. of Physics.
Woolfson, M. M., 1997, An Introduction to X-ray Crystallography, Cambridge Univ. Pr., Cambridge, UK.
Zhang, H., B. Tweel, et al. (2004). “Crystal structure of the carboxyltransferase domain of acetyl-coenzyme A carboxylase in complex with CP-640186.” Structure 12(9): 1683-91.
Zhang, H., B. Tweel, et al. (2004). “Molecular basis for the inhibition of the carboxyltransferase domain of acetyl-coenzyme-A carboxylase by haloxyfop and diclofop.” Proc Natl Acad Sci USA 101(16): 5910-5.
Zhang, H., Z. Yang, et al. (2003). “Crystal structure of the carboxyltransferase domain of acetyl-coenzyme A carboxylase.” Science 299(5615): 2064-7.

### Computer Software And Databases

AMBER, version 4.0 (P. A. Kollman, University of California at San Francisco, COPYRGT 1994).
AUTODOCK (Goodsell, D. S. and A. J. Olsen, “Automated Docking of Substrates to Proteins by Simulated Annealing” Proteins: Structure. Function, and Genetics 8:195-202 (1990), available from Scripps Research Institute, La Jolla, Calif.)
Biotechnology Software Directory, MaryAnn Liebert Publ., New York (1995).
CAVEAT (Bacon et al., J. Mol. Biol. 225:849-858 (1992))
CAVEAT (Bartlett, P. A. et al., “CAVEAT: A Program to Facilitate the Structure-Derived Design of Biologically Active Molecules.” In Molecular Recognition in Chemical and Biological Problems, Special Pub., Royal Chem. Soc., 78, pp. 82-196 (1989))
DOCK (Kuntz, I. D. et al., “A Geometric Approach to Macromolecule-Ligand Interactions,” J.-Mol. Biol. 161:269-288 (1982), available from University of California, San Francisco, Calif.)
Gaussian 92, revision C (M. J. Frisch, Gaussian, Inc., Pittsburgh, Pa., COPYRGT 1992).
GRID (Goodford, P. J., “A Computational Procedure for Determining Energetically Favorable Binding Sites on Biologically Important Macromolecules,” J. Med.

Chem. 28:849-857 (1985), available from Oxford University, Oxford, UK)
HOOK (available from Molecular Simulations, Burlington, Mass.).
Insight II/Discover (Biosysm Technologies Inc., San Diego, Calif. COPYRGT 1994).
LEGEND (Nishibata, Y. and A. Itai, Tetrahedron 47:8985 (1991), available from Molecular Simulations, Burlington, Mass.); and LeapFrog (available from Tripos Associates, St. Louis, Mo.).
LOOK from Molecular Applications Group, Developed by Dr. Christopher Lee
LUDI (Bohm, H.-J., “The Computer Program LUDI: A New Method for the De Novo Design of Enzyme Inhibitors”, J. ComR. Aid. Molec. Design, 6, pp. 61-78 (1992), available from Biosym Technologies, San Diego, Calif.)
MACCS-3D (MDL Information Systems, San Leandro, Calif. and Martin, Y. C., “3 D Database Searching in Drug Design”, J. Med. Chem. 35: 2145-2154 (1992); and
MCSS (Miranker, A. and M. Karplus, “Functionality Maps of Binding Sites: A Multiple Copy Simultaneous Search Method.” Proteins: Structure, Function and Genetics 11: 29-34 (1991), available from Molecular Simulations, Burlington, Mass.)
QUANTA/CHARMM (Molecular Simulations, Inc., Burlington, Mass. COPYRGT 1994).

LENGTHY TABLES
The patent application contains a lengthy table section. A copy of the table is available in electronic form from the USPTO web site (http://seqdata.uspto.gov/?pageRequest=docDetail&DocID=US20090155815A1). An electronic copy of the table will also be available from the USPTO upon request and payment of the fee set forth in 37 CFR 1.19(b)(3).

1. A crystal comprising a dimer of human ACC2 CT, or a fragment, or target structural motif or derivative thereof, and a ligand, wherein said ligand is a small molecule inhibitor.

2. The crystal of claim 1 wherein said fragment or derivative thereof is a peptide comprising SEQ ID NO: 6 or a peptide having at least 95% sequence identity to SEQ ID NO: 6.

3. The crystal of claim 1 wherein said crystal has a spacegroup of P2₁2₁2₁.

4. The crystal of claim 1 wherein said ligand has the following structure:

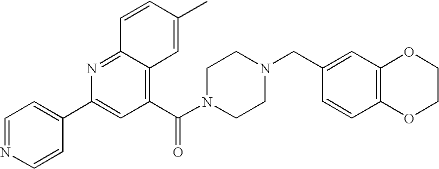

5. A crystal of claim 1 comprising an atomic structure characterized by the coordinates of Table 1.

6. The crystal of claim 1 comprising a unit cell having dimensions of about a=100.646, b=145.993, c=308.696, alpha=90.00, beta=90.00, gamma=90.00.

7. A computer system comprising: (a) a database containing information on the three dimensional structure of human ACC2 CT, or a fragment or a target structural motif or derivative thereof, and a ligand, wherein said ligand is a small molecule inhibitor, stored on a computer readable storage medium; and, (b) a user interface to view the information.

8. A computer system of claim 7, wherein the information comprises diffraction data obtained from a crystal comprising SEQ ID NO: 6.

9. A computer system of claim 7, wherein the information comprises an electron density map of a crystal form comprising SEQ ID NO: 6.

10. A computer system of claim 7, wherein the information comprises the structure coordinates of Table 1 or homologous structure coordinates comprising a root mean square deviation of non-hydrogen atoms of less than about 1.5 Å when superimposed on the non-hydrogen atom positions of the corresponding atomic coordinates of Table 1.

11. A method of identifying an agent that binds to human actyl-CoA carboxylase 2 or human actyl-CoA carboxylase 1 comprising a step of employing a three dimensional structure of human ACC2 CT that has been cocrystallized with a small molecule inhibitor.

12. A method of claim 11, wherein the three dimensional structure corresponds to the atomic structure characterized by the coordinates of Table 1 or similar structure coordinates comprising a root mean square deviation of non-hydrogen atoms of less than about 1.5 Å when superimposed on the non-hydrogen atom positions of the corresponding atomic coordinates of Table 1.

13. A method of claim 11, further comprising the steps of: synthesizing the agent; and contacting the agent with human ACC2 CT.

14. The method of claim 11, further comprising locating the attachment site of said agent to human ACC2 CT, comprising: (a) obtaining X-ray diffraction data for the crystal of human ACC2 CT; (b) obtaining X-ray diffraction data for a complex of human ACC2 CT and the agent; (c) subtracting the X-ray diffraction data obtained in step (a) from the X-ray diffraction data obtained in step (b) to obtain the difference in the X-ray diffraction data; (d) obtaining phases that correspond to X-ray diffraction data obtained in step (a); (e) utilizing the phases obtained in step (d) and the difference in the X-ray diffraction data obtained in step (c) to compute a difference Fourier image of the agent; and, (f) locating the attachment site of the agent to human ACC2 CT based on the computations obtained in step (e).

15. An isolated protein fragment comprising a binding pocket or active site defined by structure coordinates of human ACC2 CT.

16. A method for the production of a crystal complex comprising a human ACC2 CT polypeptide-ligand comprising: (a) contacting the human ACC2 CT polypeptide with said ligand in a suitable solution comprising 10% PEG 3350, 100 mM Hepes pH 7.5, 200 mM Proline, and, b) crystallizing said resulting complex of human ACC2 CT polypeptide-ligand from said solution.

17. The method of claim 11, further comprising identifying a potential inhibitor of human ACC1 or human ACC2 comprising: a) using a three dimensional structure of human ACC2 CT as defined by atomic coordinates according to Table 1; b) replacing one or more human ACC2 CT amino acids selected from A459-A462, A530-A538, B261-B270 in said three-dimensional structure with a different amino acid to produce a modified human ACC2 CT; c) using said three-dimensional structure to design or select said potential inhibitor; d) synthesizing said potential inhibitor; and, e) contacting said potential inhibitor with said modified human ACC2 CT in the presence of a substrate to test the ability of said potential inhibitor to inhibit human ACC2 CT or said modified human ACC2 CT.
